(19) United States
(12) Patent Application Publication
(10) Pub. No.: US 2020/0206506 A1
(43) Pub. Date: Jul. 2, 2020

(54) Systems and Methods for Measuring an Electrical Stapedial Reflex Threshold in a Cochlear Implant Recipient

(71) Applicant: Advanced Bionics AG, Staefa (CH)

(72) Inventors: Kurt J. Koester, Los Angeles, CA (US); Constantin Buliga, Valencia, CA (US); Kanthaiah Koka, Valencia, CA (US)

(21) Appl. No.: 16/236,303

(22) Filed: Dec. 28, 2018

(51) Int. Cl. A61N 1/36; A61N 1/05

(52) U.S. Cl. CPC A61N 1/36039; A61N 1/08; A61N 1/0541

(57) ABSTRACT

An exemplary diagnostic system directs a cochlear implant to step through applying a sequence of stimulation events each having a different stimulation level to an electrode set disposed on an electrode lead, detects, while the cochlear implant is stepping through applying the sequence of stimulation events to the electrode set, user input indicating that a stapedial reflex state within the recipient changes from a first reflex state to a second reflex state, identifies a particular stimulation event that corresponds to the change in the stapedial reflex state from the first reflex state to the second reflex state, the particular stimulation event having a particular stimulation level, and graphically presents one or more graphical markers indicating that the stapedial reflex state is the second reflex state at the particular stimulation level.

### BACKGROUND INFORMATION

When a cochlear implant is initially implanted in a recipient, and during follow-up tests and checkups thereafter, it is usually desirable to fit the cochlear implant to the recipient. Such “fitting” may include adjustment of the base amplitude or intensity of the various stimuli generated by the cochlear implant from the factory settings (or default values) to values that are most effective and comfortable for the recipient. For example, the intensity or amplitude and/or duration of the individual stimulation pulses provided by the cochlear implant may be mapped to an appropriate dynamic audio range so that the appropriate “loudness” of sensed audio signals is perceived. That is, loud sounds should be sensed by the recipient at a level that is perceived as loud, but not painfully loud. Soft sounds should similarly be sensed by the recipient at a level that is soft, but not so soft that the sounds are not perceived at all.

One aspect of fitting a cochlear implant to a particular recipient is determining at least one most comfortable level (“M level”), also known as a “most comfortable current level”. An M level refers to a stimulation current level applied by a cochlear implant at which the recipient is most comfortable. M levels typically vary from recipient to recipient and may vary from electrode channel to electrode channel in a multichannel cochlear implant.

It is often desirable to employ an objective method of determining M levels for a cochlear implant recipient. One such objective method involves increasing a current level of electrical stimulation applied by a cochlear implant to the recipient until a stapedial reflex (e.g., an involuntary muscle contraction that occurs in the middle ear in response to electrical stimulation) is elicited. The current level required to elicit a stapedial reflex within a recipient (referred to herein as an “electrical stapedius reflex threshold” or “ESRT”) may then be used by a clinician as a starting point for determining an M level corresponding to the recipient. For example, an M level for a cochlear implant recipient may be set to be substantially equal to or slightly offset from an ESRT for the cochlear implant recipient.

Unfortunately, it is often difficult, cumbersome, and time consuming to identify an ESRT for a cochlear implant recipient using conventional techniques.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 illustrates an exemplary cochlear implant system according to principles described herein.

FIG. 2 illustrates a schematic structure of the human cochlea according to principles described herein.

FIG. 3 illustrates an exemplary diagnostic system according to principles described herein.

FIG. 4 illustrates an exemplary stand-alone diagnostic system according to principles described herein.

FIG. 5 shows a base module detached from a computing module according to principles described herein.

FIGS. 6-8 depict exemplary configurations in which a diagnostic system is used to perform one or more diagnostic operations according to principles described herein.

FIGS. 9A-12 illustrate an exemplary hardware implementation of the diagnostic system of FIG. 4 according to principles described herein.

FIG. 13 illustrates an exemplary graphical user interface according to principles described herein.

FIG. 14 illustrates an exemplary stimulation event according to principles described herein.

FIGS. 15-27 illustrate exemplary graphical user interfaces according to principles described herein.

FIG. 28 illustrates an exemplary method according to principles described herein.

FIG. 29 illustrates an exemplary computing device according to principles described herein.

### DETAILED DESCRIPTION

Systems and methods for measuring an ESRT in a cochlear implant recipient are described herein. For example, a diagnostic system may 1) direct a display screen to display a graphical user interface that includes a plurality of columns corresponding to a plurality of electrodes disposed on an electrode lead implanted at least partially within a cochlea of a recipient of a cochlear implant, 2) graphically indicate, within the graphical user interface, an initial stimulation level, 3) direct the cochlear implant to step through applying a sequence of stimulation events each having a different stimulation level to an electrode set included in the plurality of electrodes, the sequence of stimulation events beginning with a first stimulation event that has the initial stimulation level, 4) detect, while the cochlear implant is stepping through applying the sequence of stimulation events to the electrode set, user input indicating that a stapedial reflex state within the recipient changes from a first reflex state to a second reflex state, 5) identify a particular stimulation event included in the sequence of stimulation events that corresponds to the change in the stapedial reflex state from the first reflex state to the second reflex state, the particular stimulation event having a particular stimulation level, and 6) graphically present, within one or more columns included in the plurality of columns and corresponding to the electrode set, one or more graphical markers indicating that the stapedial reflex state is the second reflex state at the particular stimulation level.

As used herein, a “stapedial reflex state” refers to whether a stapedial reflex occurs in response to a particular stimulation event. For example, in some scenarios (e.g., when stimulation events are being applied in order of incrementally decreasing stimulation levels), the first reflex state represents a state in which a stapedial reflex occurs in response to one or more stimulation events and the second reflex state represents a state in which the stapedial reflex does not occur in response to one or more stimulation events. In other scenarios (e.g., when stimulation events are being applied in order of incrementally increasing stimulation levels), the first reflex state represents a state in which a stapedial reflex does not occur in response to one or more stimulation event and the second reflex state represents a state in which the stapedial reflex occurs in response to one or more stimulation events.

In some examples, the systems and methods described herein are implemented by a stand-alone diagnostic system that includes a computing module and a base module configured to attach to the computing module and serve as a stand for the computing module. The computing module includes a display screen and a processor configured to direct the display screen to display a graphical user interface that includes a plurality of columns corresponding to a plurality of electrodes disposed on an electrode lead implanted at least partially within a cochlea of a recipient of a cochlear implant. The base module houses an interface unit configured to be communicatively coupled to the processor and to a cochlear implant while the base module is attached to the computing module. In this configuration, the processor may be configured to 1) graphically indicate, within the graphical user interface, an initial stimulation level, 2) direct the cochlear implant to step through applying a sequence of stimulation events each having a different stimulation level to an electrode set included in the plurality of electrodes, the sequence of stimulation events beginning with a first stimulation event that has the initial stimulation level, 3) detect, while the cochlear implant is stepping through applying the sequence of stimulation events to the electrode set, user input indicating that a stapedial reflex state within the recipient changes from a first reflex state to a second reflex state, 4) identify a particular stimulation event included in the sequence of stimulation events that corresponds to the change in the stapedial reflex state from the first reflex state to the second reflex state, the particular stimulation event having a particular stimulation level, and 5) graphically present, within one or more columns included in the plurality of columns and corresponding to the electrode set, one or more graphical markers indicating that the stapedial reflex state is the second reflex state at the particular stimulation level.

The systems and methods described herein may advantageously allow a user to more effectively, efficiently, and accurately identify ESRTs for one or more channels of a multi-channel cochlear implant (i.e., a cochlear implant configured to apply electrical stimulation by way of a plurality of electrodes disposed on an electrode lead). For example, the systems and methods described herein may allow a user to focus his or her visual attention on monitoring for a physical response of the stapedius muscle while a sequence of stimulation events are applied to an electrode set disposed on an electrode lead located within a cochlea of a recipient. To illustrate, in accordance with the systems and methods described herein, the user may continuously look through a surgical microscope at the stapedius muscle to observe when the stapedius muscle twitches or otherwise physically responds to a particular stimulation event without having to divert his or her visual attention to a display monitor, input device, or other component of a computing system being used to record ESRT data. This may allow the user to more precisely determine when an ESRT occurs, which may allow a cochlear implant system to be more accurately programmed, thereby improving operation of the cochlear implant system. Moreover, the systems and methods described herein may reduce the amount of time it takes to perform an ESRT detection procedure (e.g., by minimizing the amount of time it takes for a user to record an occurrence and/or non-occurrence of a stapedial reflex), which may be advantageous for recipients and clinicians alike. These and other advantages and benefits of the systems and methods described herein will be made apparent herein.

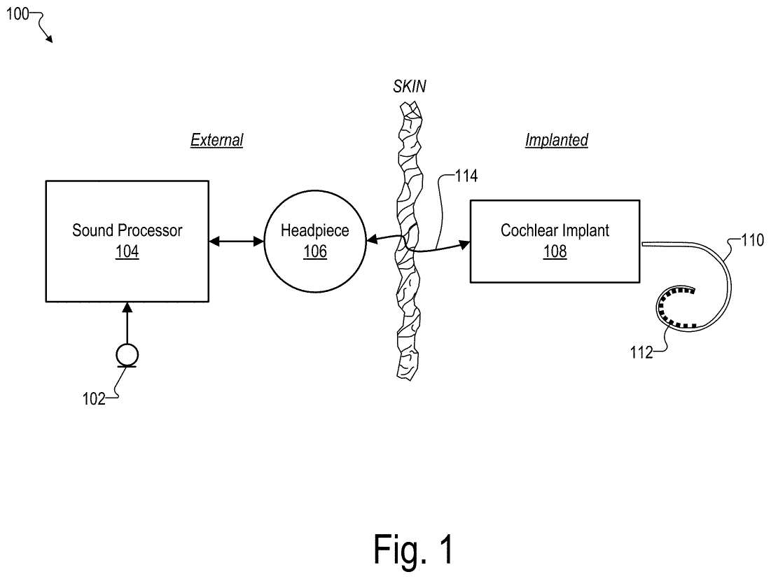

FIG. 1 illustrates an exemplary cochlear implant system 100. As shown, cochlear implant system 100 may include a microphone 102, a sound processor 104, a headpiece 106 having a coil disposed therein, a cochlear implant 108, and an electrode lead 110. Electrode lead 110 may include an array of electrodes 112 disposed on a distal portion of electrode lead 110 and that are configured to be inserted into a cochlea of a recipient to stimulate the cochlea when the distal portion of electrode lead 110 is inserted into the cochlea. One or more other electrodes (e.g., including a ground electrode, not explicitly shown) may also be disposed on other parts of electrode lead 110 (e.g., on a proximal portion of electrode lead 110) to, for example, provide a current return path for stimulation current generated by electrodes 112 and to remain external to the cochlea after electrode lead 110 is inserted into the cochlea. As shown, electrode lead 110 may be pre-curved so as to properly fit within the spiral shape of the cochlea. Additional or alternative components may be included within cochlear implant system 100 as may serve a particular implementation.

As shown, cochlear implant system 100 may include various components configured to be located external to a recipient including, but not limited to, microphone 102, sound processor 104, and headpiece 106. Cochlear implant system 100 may further include various components configured to be implanted within the recipient including, but not limited to, cochlear implant 108 and electrode lead 110.

Microphone 102 may be configured to detect audio signals presented to the user. Microphone 102 may be implemented in any suitable manner. For example, microphone 102 may include a microphone that is configured to be placed within the concha of the ear near the entrance to the ear canal, such as a T-MIC™ microphone from Advanced Bionics. Such a microphone may be held within the concha of the ear near the entrance of the ear canal during normal operation by a boom or stalk that is attached to an ear hook configured to be selectively attached to sound processor 104. Additionally or alternatively, microphone 102 may be implemented by one or more microphones disposed within headpiece 106, one or more microphones disposed within sound processor 104, one or more beam-forming microphones, and/or any other suitable microphone as may serve a particular implementation.

Sound processor 104 may be configured to direct cochlear implant 108 to generate and apply electrical stimulation (also referred to herein as “stimulation current”) representative of one or more audio signals (e.g., one or more audio signals detected by microphone 102, input by way of an auxiliary audio input port, input by way of a clinician's programming interface (CPI) device, etc.) to one or more stimulation sites associated with an auditory pathway (e.g., the auditory nerve) of the recipient. Exemplary stimulation sites include, but are not limited to, one or more locations within the cochlea, the cochlear nucleus, the inferior colliculus, and/or any other nuclei in the auditory pathway. To this end, sound processor 104 may process the one or more audio signals in accordance with a selected sound processing strategy or program to generate appropriate stimulation parameters for controlling cochlear implant 108. Sound processor 104 may be housed within any suitable housing (e.g., a behind-the-ear (“BTE”) unit, a body worn device, headpiece 106, and/or any other sound processing unit as may serve a particular implementation).

In some examples, sound processor 104 may wirelessly transmit stimulation parameters (e.g., in the form of data words included in a forward telemetry sequence) and/or power signals to cochlear implant 108 by way of a wireless communication link 114 between headpiece 106 and cochlear implant 108 (e.g., a wireless link between a coil disposed within headpiece 106 and a coil physically coupled to cochlear implant 108). It will be understood that communication link 114 may include a bi-directional communication link and/or one or more dedicated uni-directional communication links.

Headpiece 106 may be communicatively coupled to sound processor 104 and may include an external antenna (e.g., a coil and/or one or more wireless communication components) configured to facilitate selective wireless coupling of sound processor 104 to cochlear implant 108. Headpiece 106 may additionally or alternatively be used to selectively and wirelessly couple any other external device to cochlear implant 108. To this end, headpiece 106 may be configured to be affixed to the recipient's head and positioned such that the external antenna housed within headpiece 106 is communicatively coupled to a corresponding implantable antenna (which may also be implemented by a coil and/or one or more wireless communication components) included within or otherwise associated with cochlear implant 108. In this manner, stimulation parameters and/or power signals may be wirelessly transmitted between sound processor 104 and cochlear implant 108 via communication link 114.

Cochlear implant 108 may include any suitable type of implantable stimulator. For example, cochlear implant 108 may be implemented by an implantable cochlear stimulator. Additionally or alternatively, cochlear implant 108 may include a brainstem implant and/or any other type of cochlear implant that may be implanted within a recipient and configured to apply stimulation to one or more stimulation sites located along an auditory pathway of a recipient.

In some examples, cochlear implant 108 may be configured to generate electrical stimulation representative of an audio signal processed by sound processor 104 (e.g., an audio signal detected by microphone 102) in accordance with one or more stimulation parameters transmitted thereto by sound processor 104. Cochlear implant 108 may be further configured to apply the electrical stimulation to one or more stimulation sites (e.g., one or more intracochlear regions) within the recipient via electrodes 112 disposed along electrode lead 110. In some examples, cochlear implant 108 may include a plurality of independent current sources each associated with a channel defined by one or more of electrodes 112. In this manner, different stimulation current levels may be applied to multiple stimulation sites simultaneously by way of multiple electrodes 112.

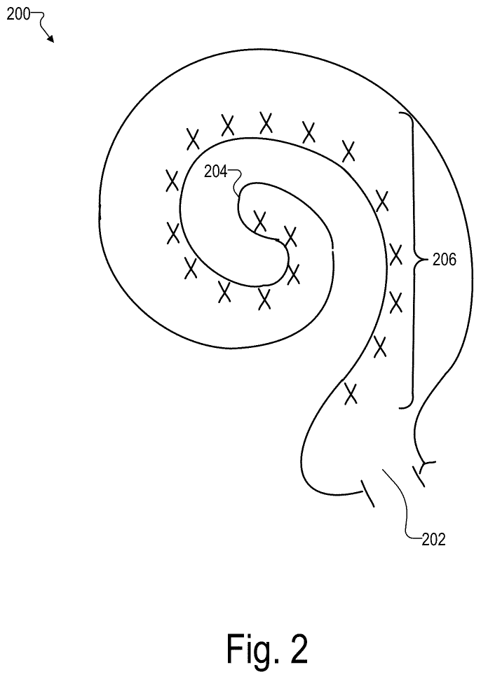

FIG. 2 illustrates a schematic structure of the human cochlea 200 into which electrode lead 110 may be inserted. As shown in FIG. 2, cochlea 200 is in the shape of a spiral beginning at a base 202 and ending at an apex 204. Within cochlea 200 resides auditory nerve tissue 206, which is denoted by Xs in FIG. 2. The auditory nerve tissue 206 is organized within the cochlea 200 in a tonotopic manner. Relatively low frequencies are encoded at or near the apex 204 of the cochlea 200 (referred to as an “apical region”) while relatively high frequencies are encoded at or near the base 202 (referred to as a “basal region”). Hence, electrical stimulation applied by way of electrodes disposed within the apical region (i.e., “apical electrodes”) may result in the recipient perceiving relatively low frequencies and electrical stimulation applied by way of electrodes disposed within the basal region (i.e., “basal electrodes”) may result in the recipient perceiving relatively high frequencies. The delineation between the apical and basal electrodes on a particular electrode lead may vary depending on the insertion depth of the electrode lead, the anatomy of the recipient's cochlea, and/or any other factor as may serve a particular implementation.

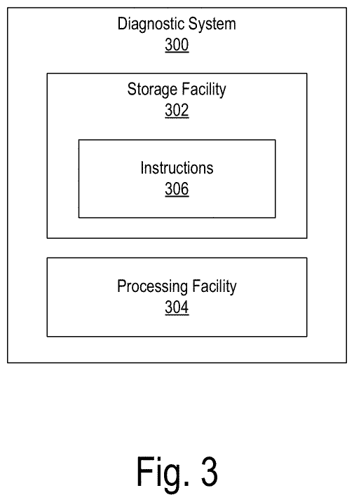

FIG. 3 illustrates an exemplary diagnostic system 300 that may be configured to perform any of the operations described herein. As shown, diagnostic system 300 may include, without limitation, a storage facility 302 and a processing facility 304 selectively and communicatively coupled to one another. Facilities 302 and 304 may each include or be implemented by hardware and/or software components (e.g., processors, memories, communication interfaces, instructions stored in memory for execution by the processors, etc.). In some examples, facilities 302 and 304 may be distributed between multiple devices and/or multiple locations as may serve a particular implementation.

Storage facility 302 may maintain (e.g., store) executable data used by processing facility 304 to perform any of the operations described herein. For example, storage facility 302 may store instructions 306 that may be executed by processing facility 304 to perform any of the operations described herein. Instructions 306 may be implemented by any suitable application, software, code, and/or other executable data instance. Storage facility 302 may also maintain any data received, generated, managed, used, and/or transmitted by processing facility 304.

Processing facility 304 may be configured to perform (e.g., execute instructions 306 stored in storage facility 302 to perform) various operations associated with measuring an ESRT for a cochlear implant recipient. For example, processing facility 304 may 1) direct a display screen to display a graphical user interface that includes a plurality of columns corresponding to a plurality of electrodes disposed on an electrode lead implanted at least partially within a cochlea of a recipient of a cochlear implant, 2) graphically indicate, within the graphical user interface, an initial stimulation level, 3) direct the cochlear implant to step through applying a sequence of stimulation events each having a different stimulation level to an electrode set included in the plurality of electrodes, the sequence of stimulation events beginning with a first stimulation event that has the initial stimulation level, 4) detect, while the cochlear implant is stepping through applying the sequence of stimulation events to the electrode set, user input indicating that a stapedial reflex state within the recipient changes from a first reflex state to a second reflex state, 5) identify a particular stimulation event included in the sequence of stimulation events that corresponds to the change in the stapedial reflex state from the first reflex state to the second reflex state, the particular stimulation event having a particular stimulation level, and 6) graphically present, within one or more columns included in the plurality of columns and corresponding to the electrode set, one or more graphical markers indicating that the stapedial reflex state is the second reflex state at the particular stimulation level. These and other operations that may be performed by processing facility 304 are described in more detail herein.

Diagnostic system 300 may be implemented in any suitable manner. For example, diagnostic system 300 may be implemented by a stand-alone diagnostic system that may be used in or outside a surgical operating room to perform any of the operations described herein.

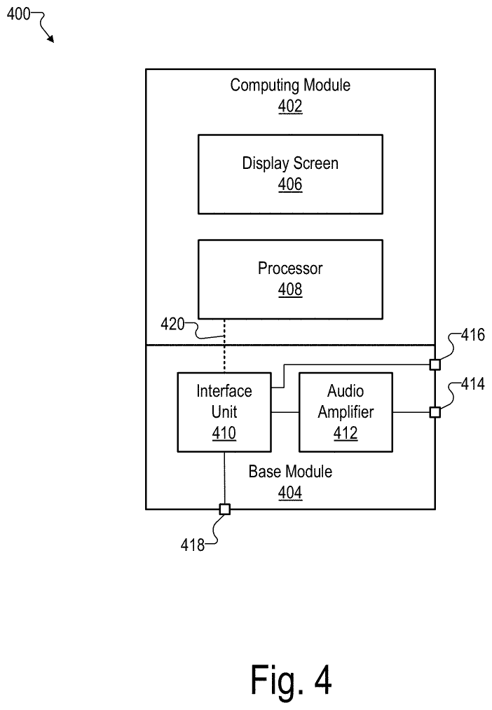

FIG. 4 illustrates an exemplary stand-alone diagnostic system 400 that may implement diagnostic system 300. As shown, diagnostic system 400 includes a computing module 402 and a base module 404. Computing module 402 includes a display screen 406 and a processor 408. Base module 404 includes an interface unit 410, an audio amplifier 412, an audio output port 414, a communications port 416, and a port 418. Computing module 402 and base module 404 may include additional or alternative components as may serve a particular implementation. For example, computing module 402 and/or base module 404 may include one or more speakers configured to output acoustic feedback and/or other types of sound configured to be heard by a surgeon and/or other user of diagnostic system 400. Diagnostic system 400 and exemplary implementations thereof are described more fully in co-pending PCT Application No. PCT/US18/67900, which application is filed the same day as the present application and incorporated herein by reference in its entirety.

In the configuration shown in FIG. 4, base module 404 is physically attached to computing module 402. In this configuration, processor 408 is communicatively coupled to interface unit 410 by way of a connection 420. Connection 420 may be implemented by any suitable connection (e.g., an internal USB connection) as may serve a particular implementation. As will be described in more detail below, base module 404 may be selectively detached from computing module 402 and connected to a different computing device by way of port 418.

Display screen 406 may be configured to display any suitable content associated with an application executed by processor 408. Display screen 406 may be implemented by a touchscreen and/or any other type of display screen as may serve a particular implementation.

Processor 408 may be configured to execute a diagnostic application associated with a cochlear implant (e.g., cochlear implant 108). For example, processor 408 may execute a diagnostic application that may be used during a procedure (e.g., an intraoperative or postoperative procedure) associated with the cochlear implant. The diagnostic application may be configured to perform various diagnostic operations associated with the cochlear implant during the procedure. Exemplary diagnostic operations are described herein.

In some examples, processor 408 may direct display screen 406 to display a graphical user interface associated with the diagnostic application being executed by processor 408. A user may interact with the graphical user interface to adjust one or more parameters associated with the cochlear implant and/or otherwise obtain information that may be useful during a procedure associated with the cochlear implant.

Base module 404 may be configured to attach to computing module 402 and serve as a stand for computing module 402.

Interface unit 410 is configured to be communicatively coupled to processor 408 by way of connection 420 while base module 404 is attached to computing module 402. Interface unit 410 is further configured to be communicatively coupled to the cochlear implant while base module 404 is attached to computing module 402. In this manner, interface unit 410 provides an interface between processor 408 and the cochlear implant.

Interface unit 410 may be communicatively coupled to the cochlear implant by way of communications port 416. For example, communications port 416 may be selectively coupled to a coil (e.g., a coil included in a headpiece, such as headpiece 106, or a disposable stand-alone coil) configured to wirelessly communicate with the cochlear implant. Interface unit 410 may communicate with the cochlear implant by transmitting and/or receiving data to/from the cochlear implant by way of the coil connected to communications port 416.

Interface unit 410 may be further configured to generate and provide acoustic stimulation (e.g., sound waves) to the recipient of the cochlear implant. To this end, audio output port 414 is configured to be selectively coupled to a sound delivery apparatus. In some examples, the sound delivery apparatus may be implemented by tubing that has a distal portion configured to be placed in or near an entrance to an ear canal of a recipient of the cochlear implant. While the sound delivery apparatus is connected to audio output port 414, interface unit 410 may transmit the acoustic stimulation to the recipient by way of the sound delivery apparatus.

As shown, audio amplifier 412 may be positioned within a path between interface unit 410 and audio output port 414. In this configuration, audio amplifier 412 may be configured to amplify the acoustic stimulation before the acoustic stimulation is delivered to the recipient by way of audio output port 414 and the sound delivery apparatus. In some alternative examples, amplification of the acoustic stimulation generated by interface unit 410 is not necessary, thereby obviating the need for audio amplifier 412 to be included in base module 404. Hence, in some implementations, base module 404 does not include audio amplifier 412.

In some examples, diagnostic system 400 may be configured to self-calibrate and/or perform in-situ testing. For example, processor 408 may calibrate an amplitude level of acoustic stimulation generated by interface unit 410 before and/or during a procedure in which diagnostic system 400 is used to perform any of the operations described herein. Such self-calibration and in-situ testing may be performed in any suitable manner.

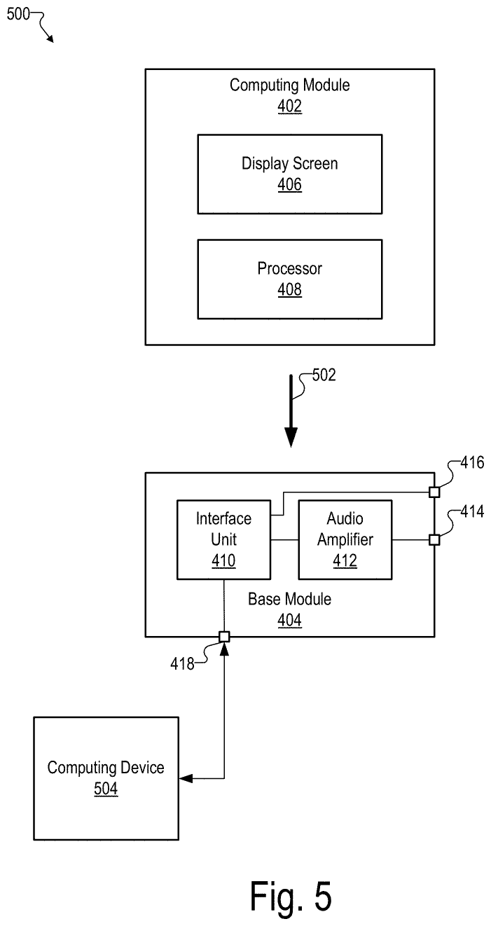

As mentioned, base module 404 may be selectively detached from computing module 402. To illustrate, FIG. 5 shows a configuration 500 in which base module 404 is detached from computing module 402. This detachment is illustrated by arrow 502. While detached, interface unit 410 of base module 404 may be communicatively coupled to a computing device 504. For example, interface unit 410 may be communicatively coupled to computing device 504 by plugging a cable (e.g., a USB cable) into port 418 and into computing device 504. In this configuration, computing device 504 may use interface unit 410 to interface with a cochlear implant (e.g., by providing acoustic stimulation to a recipient of the cochlear implant and/or receiving recording data from the cochlear implant).

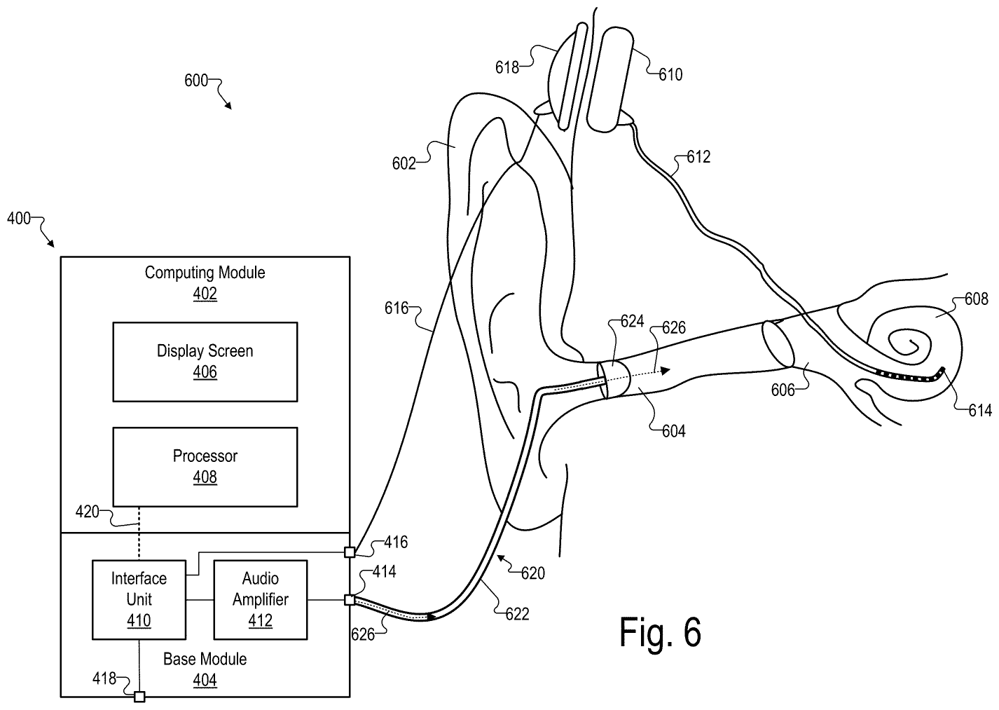

FIG. 6 depicts an exemplary configuration 600 in which diagnostic system 400 is used to perform one or more diagnostic operations with respect to a recipient of a cochlear implant. Various anatomical features of the recipient's ear are shown in FIG. 6. Specifically, anatomical features include a pinna 602 (i.e., the outer ear), an ear canal 604, a middle ear 606, and a cochlea 608. While no specific incision or other explicit surgical representation is shown in FIG. 6, it will be understood that such elements may be present when a procedure is ongoing. For example, an incision may be present to allow the surgeon internal access to the recipient to insert the lead into cochlea 608. In some procedures, pinna 602 may be taped down and covered with surgical drapes so as to cover ear canal 604 (e.g., to help prevent fluids from reaching ear canal 604).

In the example of FIG. 6, a cochlear implant 610 and an electrode lead 612 are shown to be implanted within the recipient. Cochlear implant 610 may be similar, for example, to cochlear implant 108, and electrode lead 612 may be similar, for example, to electrode lead 110. Electrode lead 612 includes a plurality of electrodes (e.g., electrode 614, which is the distal-most electrode disposed on electrode lead 612).

As shown, a cable 616 of a headpiece 618 is connected to communications port 416. In this configuration, interface unit 410 may wirelessly communicate with cochlear implant 610 by way a coil and/or other electronics included in headpiece 618, which may be similar to headpiece 106.

As also shown, a sound delivery apparatus 620 is connected to audio output port 414. Sound delivery apparatus 620 includes tubing 622 and an ear insert 624. Ear insert 624 is configured to fit at or within an entrance of ear canal 604. Tubing 622 and ear insert 624 together form a sound propagation channel 626 that delivers acoustic stimulation provided by interface unit 410 to the ear canal 604. Tubing 622 and ear insert 624 may be made out of any suitable material as may serve a particular implementation.

In some examples, processor 408 may execute a diagnostic application. In accordance with the diagnostic application, processor 408 may transmit, by way of connection 420, a command (also referred to as a stimulation command) to interface unit 410 for interface unit 410 to apply acoustic stimulation to the recipient. In response to receiving the command, interface unit 410 may generate and apply the acoustic stimulation to the recipient by way of audio output port 414 and sound delivery apparatus 620.

As another example, in accordance with the diagnostic application, processor 408 may transmit, by way of connection 420, a command to interface unit 410 for interface unit 410 to direct cochlear implant 610 to apply electrical stimulation to the recipient by way of one or more electrodes included on electrode lead 612. In response to receiving the command, interface unit 410 may transmit a command to cochlear implant 610 for cochlear implant 610 to generate and apply the electrical stimulation to the recipient by way of the one or more electrodes.

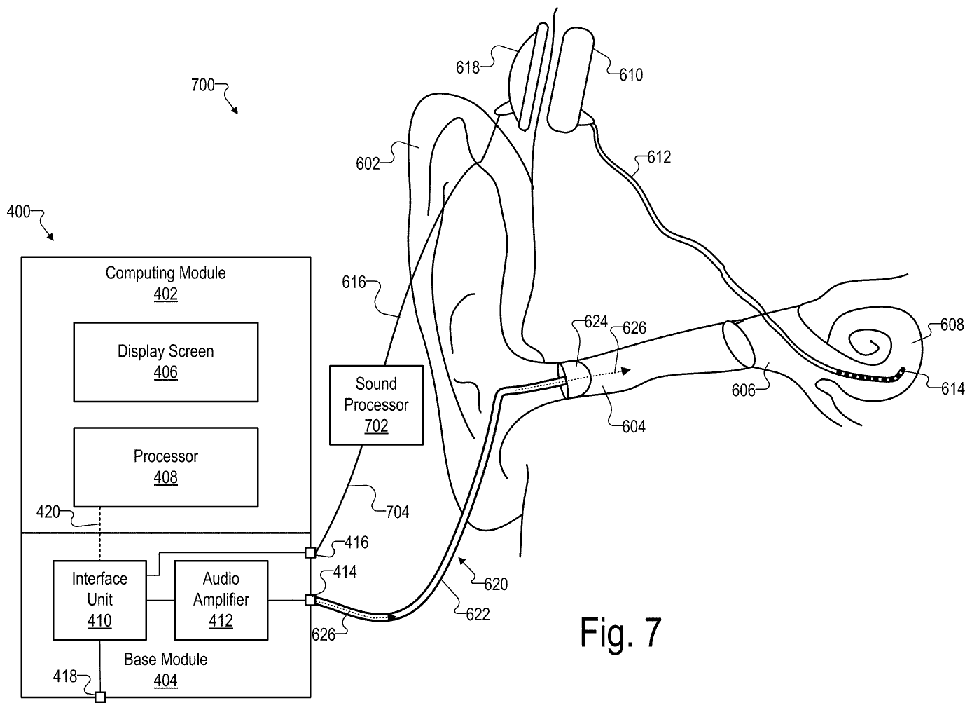

In configuration 600, headpiece 618 is connected directly to communications port 416 by way of cable 616. Hence, in configuration 600, interface unit 410 is configured to directly control cochlear implant 610. FIG. 7 illustrates an alternative configuration 700 in which a sound processor 702 is included in the communication path in between interface unit 410 and cochlear implant 610. Sound processor 702 may be similar to any of the sound processors (e.g., sound processor 104) described herein. In some examples, sound processor 702 is recipient-agnostic. In other words, sound processor 702 is not configured specifically for the recipient of cochlear implant 610. Rather, sound processor 702 may be used in a variety of different procedures associated with a number of different recipients.

As shown, sound processor 702 is connected to communications port 416 by way of a cable 704. Sound processor 702 is also connected to headpiece 618 by way of cable 616. In this configuration, sound processor 702 may relay data and/or commands between interface unit 410 and cochlear implant 610.

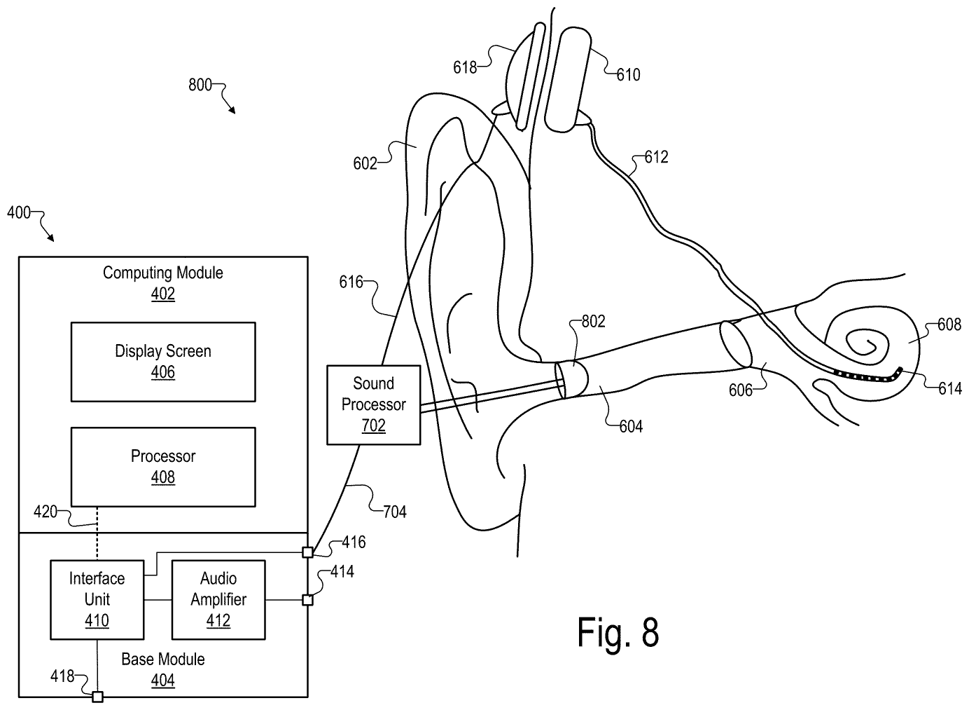

FIG. 8 illustrates an alternative configuration 800 in which sound processor 702 is configured to generate the acoustic stimulation that is applied to the recipient of cochlear implant 610. As shown, in this configuration, a sound delivery apparatus 802 is coupled directly to sound processor 702. For example, sound processor 702 may be implemented by a behind-the-ear bimodal sound processor and sound delivery apparatus 802 may be implemented by an audio ear hook that connects to sound processor 702.

It will be recognized that diagnostic system 400 may be additionally or alternatively implemented in any other suitable manner. For example, diagnostic system 400 may be implemented by a fitting system utilized in a clinician's office and/or by any other appropriately configured system or device.

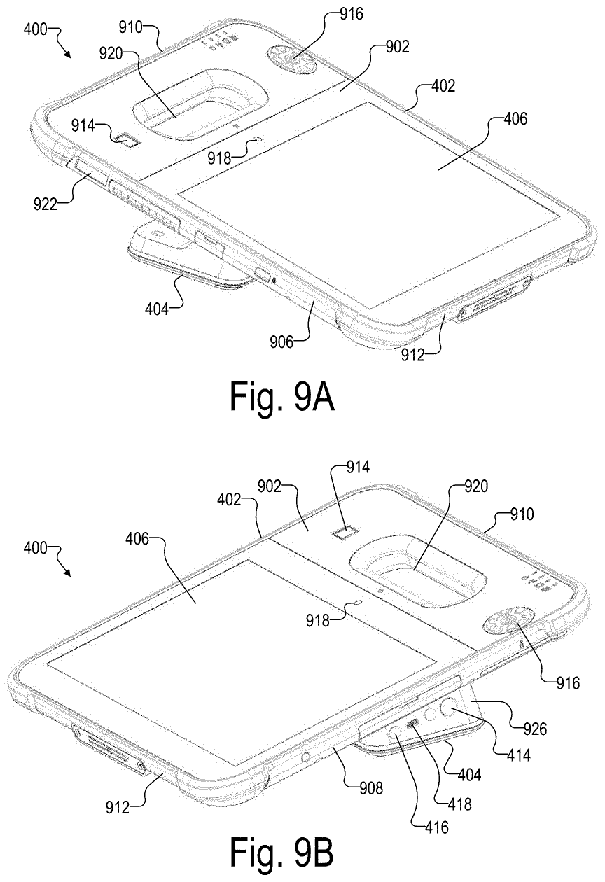

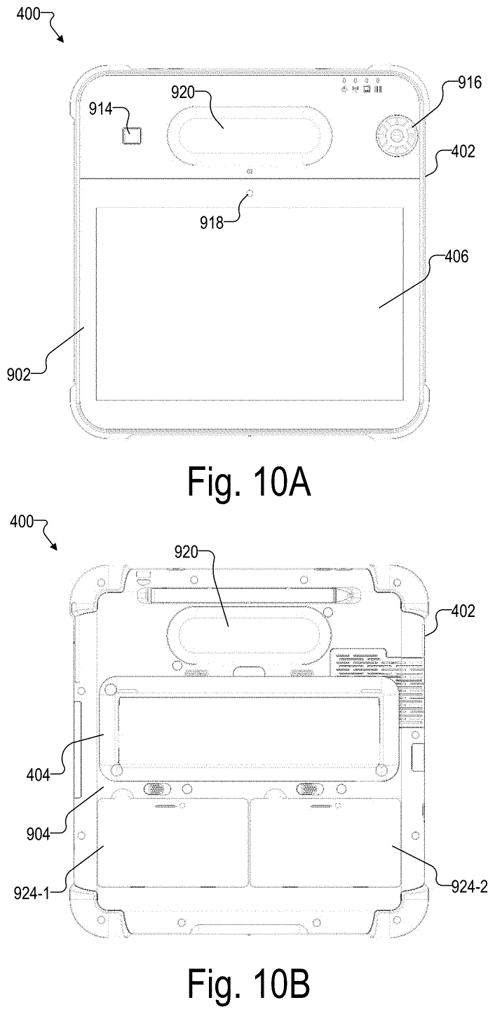

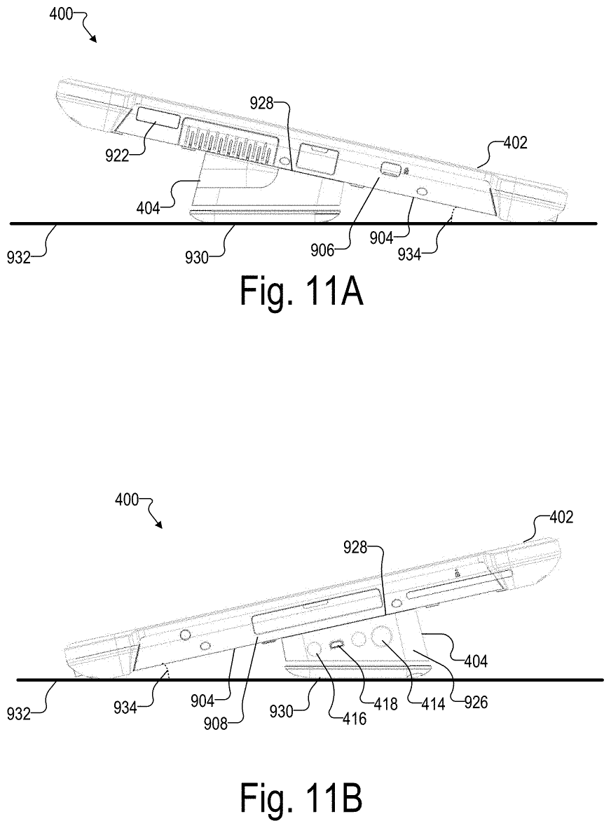

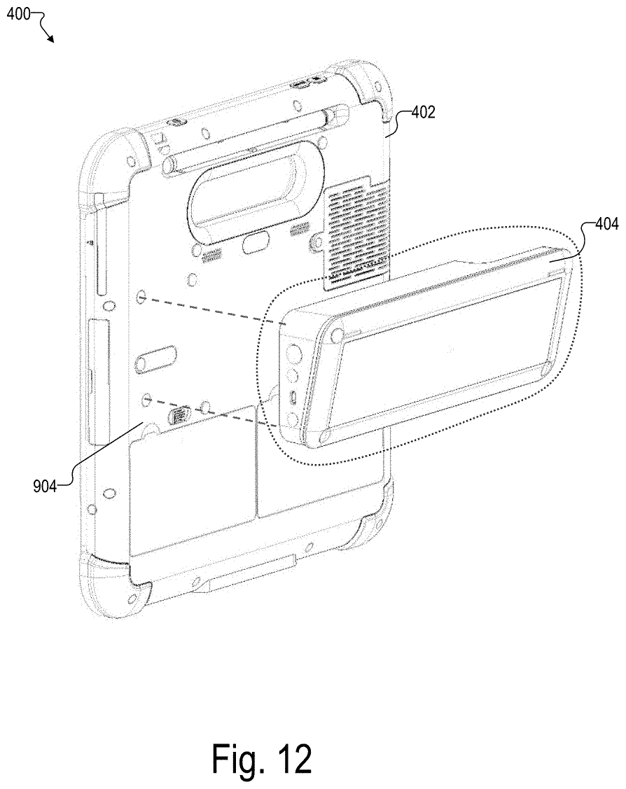

An exemplary hardware implementation of diagnostic system 400 will now be described in connection with FIGS. 9A-12. In particular, FIG. 9A shows a left perspective view of diagnostic system 400, FIG. 9B shows a right perspective view of diagnostic system 400, FIG. 10A shows a front view of diagnostic system 400, FIG. 10B shows a back view of diagnostic system 400, FIG. 11A shows a left side view of diagnostic system 400, FIG. 11B shows a right side view of diagnostic system 400, and FIG. 12 shows a rear perspective view of diagnostic system 400.

The hardware implementation of diagnostic system 400 illustrated in FIGS. 9A-12 includes computing module 402 and base module 404. As, illustrated computing module 402 includes a front side 902, a back side 904, a left side 906, a right side 908, a top side 910, and a bottom side 912.

Display screen 406 is located on front side 902 of computing module 402. Various other components are also located on the front side 902 of computing module 402. For example, a fingerprint scanner 914, physical input buttons 916, and a webcam 918 all shown to be included on the front side 902 of computing module 402. It will be recognized that any of these components may be located on any other side of computing module 402 as may serve a particular implementation.

Fingerprint scanner 914 is configured to facilitate authentication of a user of diagnostic system 400. For example, fingerprint scanner 914 may detect a fingerprint of the user and provide processor 408 with data representative of the fingerprint. Processor 408 may process the fingerprint data in any suitable manner (e.g., by comparing the fingerprint to known fingerprints included in a database) to authenticate the user.

Webcam 918 may be configured to facilitate video communication by a user of diagnostic system 400 with a remotely located user (e.g., during or after a surgical procedure). Such video communication may be performed in any suitable manner.

Physical input buttons 916 may be implemented, for example, by a directional pad and/or any other suitable type of physical input button. A user of diagnostic system 400 may interact with physical input buttons 916 to perform various operations with respect to a diagnostic application being executed by processor 408. For example, the user may use the physical input buttons 916 to interact with a graphical user interface displayed on display screen 406.

In some examples, physical input buttons 916 may be configured to be selectively programmed (e.g., as hotkeys) to perform one or more functions associated with the diagnostic application. For example, a particular physical input button 916 may be programmed by a user to start and/or stop acoustic stimulation being applied to a cochlear implant recipient by diagnostic system 400.

In some examples, processor 408 may be configured to wirelessly connect to an input device configured to be used by the user in connection with the diagnostic application. For example, processor 408 may be configured to wirelessly connect (e.g., via Bluetooth and/or any other suitable wireless communication protocol) to a keyboard, mouse, remote control, and/or any other wireless input device as may serve a particular implementation. In this manner, the user may selectively use physical input buttons 916, a touchscreen capability of display screen 406, and/or a wireless input device to interact with diagnostic system 400.

As shown, a hole 920 may be formed within computing module 402 and configured to serve as a handle for diagnostic system 400. A user may grip computing module 402 by placing his or her fingers within hole 920.

As shown, a barcode scanner 922 may be located on left side 906 of computing module 402. Barcode scanner 922 may alternatively be located on any other side of computing module 402. In some examples, barcode scanner 922 may be configured to scan for an activation code included on one or more components associated with a procedure being performed with respect to cochlear implant 510. The activation code may be used to associate (e.g., register) the components with cochlear implant 510.

As illustrated in FIG. 10B, computing module 402 may include batteries 924-1 and 924-2. Batteries 924 may be configured to provide operating power for various components included within computing module 402 and base module 404. In some examples, batteries 924 may be hot-swappable. In other words, one of batteries 924 (e.g., battery 924-1) may be removed and replaced while the other battery (e.g., battery 924-2) is used to provide power to computing module 402 and base module 404.

As illustrated in FIGS. 9B and 11B, ports 414, 416, and 418 are located on a side surface 926 of base module 404. Ports 414, 416, and 418 may alternatively be located on any other surface of base module 404.

As described above, base module 404 may be configured to serve as a stand for computing module 402 while base module 404 is attached to computing module 402. The stand functionality of base module 404 is illustrated in FIGS. 11A-11B.

As shown, base module 404 includes a top surface 928 configured to selectively attach to back side 904 of computing module 402. Base module 404 may alternatively attach to any other side of computing module 402. Base module 404 further includes a bottom surface 930 configured to be placed on a resting surface 932. Bottom surface 930 is angled with respect to back side 904 of computing module 402. This provides a viewing angle 934 for display screen 406 that is greater than zero degrees with respect to resting surface 932. In some examples, base module 404 may be adjustable to selectively provide different viewing angles for display screen 406 with respect to resting surface 932. This adjustability may be realized in any suitable manner. For example, a user may manually adjust bottom surface 930 to different angles with respect to back side 904 of computing module 402.

FIG. 12 illustrates an exemplary configuration in which base module 404 is detached from computing module 402. Base module 404 may be detached from computing module 402 in any suitable manner. For example, base module 404 may include one or more locking mechanisms that may be actuated by a user to detach base module 404 from computing module 402.

Various operations that may be performed by diagnostic system 300 will now be described. It will be recognized that diagnostic system 300 may perform additional or alternative operations to those described herein as may serve a particular implementation.

As mentioned, diagnostic system 300 may direct a display screen to display a graphical user interface that includes a plurality of columns corresponding to a plurality of electrodes disposed on an electrode lead implanted at least partially within a cochlea of a recipient of a cochlear implant. The display screen may be similar to or implemented by any of the display screens described herein. Diagnostic system 300 may direct the display screen to display the graphical user interface in accordance with a diagnostic application being executed by diagnostic system 300.

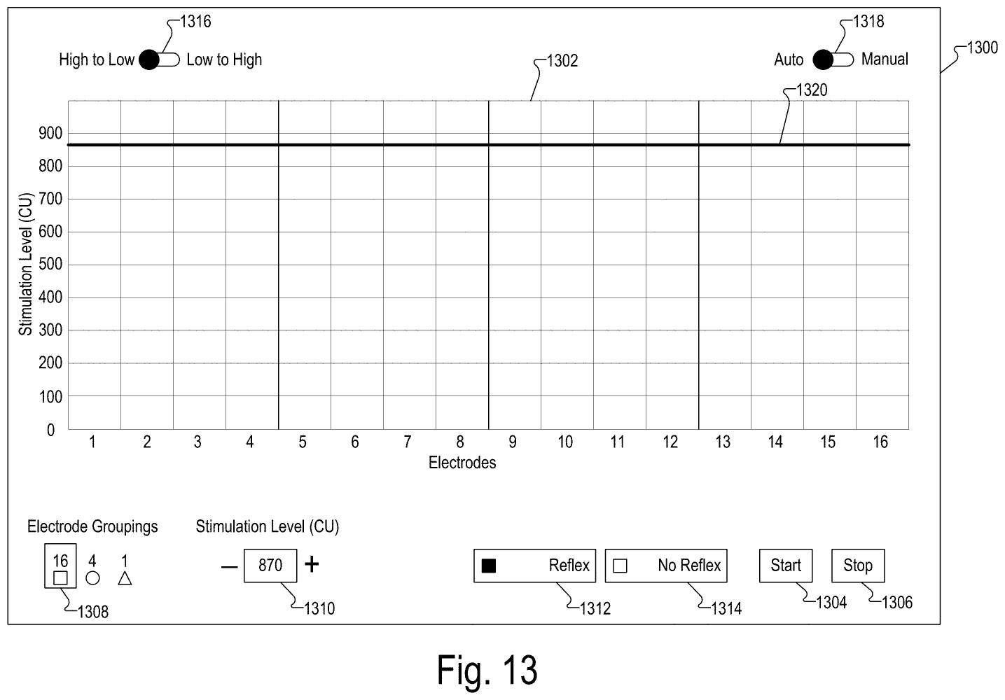

FIG. 13 illustrates an exemplary graphical user interface 1300 that may be presented by diagnostic system 300 by way of a display screen. As shown, graphical user interface 1300 may include a graph 1302, a start option 1304, a stop option 1306, an electrode grouping selector 1308, a stimulation level adjustment field 1310, a reflex indicator option 1312, a no reflex indicator option 1314, a stimulation event sequence direction selector 1316, and an auto/manual selector 1318. Graphical user interface 1300 may include additional or alternative display elements as may serve a particular implementation.

As shown, each column along an x-axis of graph 1302 is labeled with and corresponds to a particular electrode number. Each electrode number represents an electrode disposed on an electrode lead that has been at least partially implanted within a cochlea of a recipient of a cochlear implant. In the examples provided herein, it will be assumed that sixteen electrodes are disposed on the electrode lead. The most apical electrode (i.e., the electrode that is most distally located on the electrode lead) is labeled “1” in graph 1302. The most basal electrode (i.e., the electrode that is most proximately located on the electrode lead) is labeled “16” in graph 1302. It will be recognized that any number of electrodes may be disposed on the electrode lead as may serve a particular implementation. It will also be recognized that while the columns within graph 1302 are vertically oriented, they may alternatively be horizontally oriented.

The y-axis in graph 1302 represents various stimulation levels of stimulation events that may be applied to an electrode set included in the electrodes disposed on the electrode lead.

Diagnostic system 300 may graphically indicate, within graphical user interface 1300, an initial stimulation level. For example, in graphical user interface 1300, the initial stimulation level is 870 clinical units (CU) and is graphically indicated by a horizontal stimulation level indicator 1320 within graph 1302 and by stimulation level adjustment field 1310. It will be recognized that the initial stimulation level may alternatively be graphically indicated by any other suitable graphical object. In some examples, a user may reposition stimulation level indicator 1320 (e.g., by graphically dragging stimulation level indicator 1320 to a different position within graph 1302). Diagnostic system 300 may detect this repositioning and adjust the initial stimulation level based on the repositioning. For example, in response to a user repositioning stimulation level indicator 1320 to a stimulation level of 700 CU, diagnostic system 300 may set the initial stimulation level to be equal to 700 CU.

The initial stimulation level represents a stimulation level at which a cochlear implant within a recipient is to begin stepping through applying a sequence of stimulation events to an electrode set included within the plurality of electrodes disposed on the electrode lead. As used herein, a stimulation event includes one or more periods of electrical stimulation applied at a particular stimulation level. For example, as will be described herein, a stimulation event may include a set number (e.g., three) of temporally spaced periods of electrical stimulation each applied at the same stimulation level.

The electrode set to which the sequence of stimulation events is applied may include any number of electrodes disposed on the electrode lead. In some examples, a user may interact with graphical user interface 1300 to select electrodes for inclusion in the electrode set. For example, the user may position electrode grouping selector 1308 over a preset electrode grouping number (e.g., sixteen electrodes, four electrodes, or one electrode) to indicate how many electrodes are to be included in the electrode set. In the example of FIG. 13, electrode grouping selector 1308 is positioned over an electrode grouping number of sixteen, thereby indicating that all sixteen electrodes disposed on the electrode lead are to be included in the electrode set to which stimulation events are applied. If the user repositions electrode grouping selector 1308 to an electrode grouping number of four or one, the user may select either four or one electrodes for inclusion in the electrode set (e.g., by selecting columns that correspond to the desired electrode numbers). An example of this will be provided herein.

Stimulation event sequence direction selector 1316 is configured to set a direction (e.g., high to low or low to high) of the stimulation event sequence that is applied by the cochlear implant to the electrode set. For example, in FIG. 13, stimulation event sequence direction selector 1316 is set to high to low. Based on this setting, diagnostic system 300 may direct the cochlear implant to apply a plurality of stimulation events to the electrode set in order of incrementally decreasing stimulation levels. Alternatively, if stimulation event sequence direction selector 1316 is set to low to high, diagnostic system 300 may direct the cochlear implant to apply a plurality of stimulation events to the electrode set in order of incrementally increasing stimulation levels. Examples of this will be provided herein.

Auto/manual selector 1318 is configured to set a manner in which the stimulation events are applied to the electrode set. In the example of FIG. 13, auto/manual selector 1318 is set to auto. In this configuration, as will be described herein, diagnostic system 300 may direct the cochlear implant to automatically step through applying the sequence of stimulation events to the electrode set without waiting for user input after each stimulation event is applied. Alternatively, if auto/manual selector 1318 is set to manual, user input representative of a request to apply an additional stimulation event is required after each stimulation event is applied. In the examples provided herein, it will be assumed that auto/manual selector 1318 is set to auto.

In response to a selection by a user of start option 1304, diagnostic system 300 may direct the cochlear implant to step through applying a sequence of stimulation events each having a different stimulation level to the electrode set. At any point during the application of the stimulation events, the user may select stop option 1306 to stop the application of the sequence of events.

The sequence of stimulation events begins with a first stimulation event that has the initial stimulation level. Depending on the sequence direction set by stimulation event sequence direction selector 1316, diagnostic system 300 may direct the cochlear implant to either incrementally increase the stimulation level of each subsequent stimulation event included in the sequence of stimulation events or incrementally decrease the stimulation level of each subsequent stimulation event included in the sequence of stimulation events. Examples of both sequence directions will be provided herein.

While the cochlear implant is stepping through applying the sequence of stimulation events to the electrode set, the user may visually monitor for a stapedial reflex that occurs in response to one or more of the stimulation events. For example, as described above, the user may look through a surgical microscope at the stapedius muscle to observe when the stapedius muscle twitches or otherwise physically responds to a particular stimulation event. If the user detects a change in a stapedial reflex state from a first reflex state to a second reflex state, the user may provide user input indicative of the change. For example, the user may select option 1312 or option 1314, depending on the nature of the change.

Diagnostic system 300 may detect the user input and, in response, identify a particular stimulation event included in the sequence of stimulation events that corresponds to the change in the stapedial reflex state from the first reflex state to the second reflex state. Diagnostic system 300 may graphically present, within one or more columns that correspond to the electrode set, one or more graphical markers indicating that the stapedial reflex state is the second reflex state at a stimulation level of the stimulation event.

Various examples will now be provided. It will be recognized that the examples provided herein are merely illustrative of the many different manners in which the systems and methods described herein may be used to facilitate measurement of an ESRT in a cochlear implant recipient.

FIGS. 13-20 illustrate an exemplary ESRT measurement session that automatically and sequentially steps through a plurality of stimulation events having incrementally decreasing stimulation levels. With reference to FIG. 13, a user may select start option 1304 to initiate the ESRT measurement session. In response to the user selection of start option 1304, diagnostic system 300 directs the cochlear implant to apply (e.g., concurrently) a stimulation event to an electrode set that includes all sixteen electrodes disposed on an electrode lead that is implanted within a cochlea of a recipient. The stimulation event has the initial stimulation level graphically indicated by stimulation level indicator 1320 and stimulation level adjustment field 1310. In the particular example of FIG. 13, this initial stimulation level is 870 CU.

Diagnostic system 300 may direct the cochlear implant to apply the stimulation event in any suitable manner. For example, diagnostic system 300 may transmit a command to a sound processor (e.g., sound processor 702) associated with the cochlear implant. The command may direct the sound processor to transmit a command to the cochlear implant for the cochlear implant to apply the stimulation event to the electrode set.

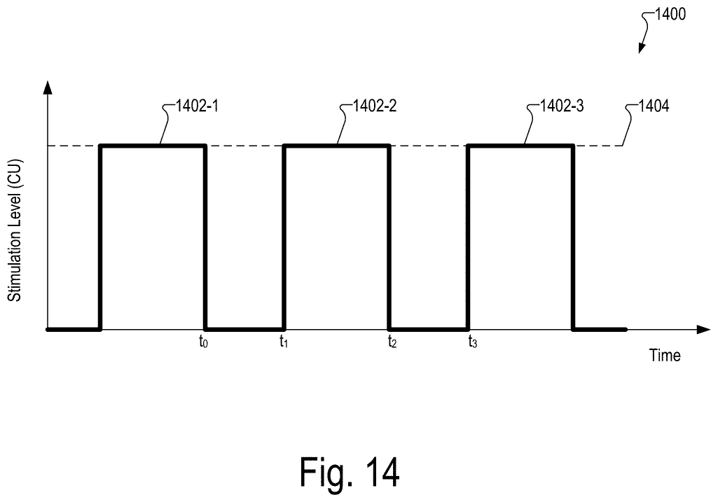

FIG. 14 illustrates an exemplary stimulation event 1400 that may be applied by the cochlear implant to the electrode set. As shown, stimulation event 1400 includes a set number of temporally spaced periods of electrical stimulation 1402-1 through 1402-3. In the example of FIG. 14, stimulation event 1400 includes three periods of electrical stimulation 1402. The first and second periods of electrical stimulation 1402-1 and 1402-2 are temporally spaced apart by a time period equal to t₁-t₀. The second and third periods of electrical stimulation 1402-2 and 1402-3 are temporally spaced apart by a time period equal to t₃-t₂. This temporal spacing may be of any suitable duration. For example, the periods of electrical stimulation 1402 may be temporally spaced apart from one another by 0.5-1.0 seconds or any other amount of time.

Each period of electrical stimulation 1402 may include one or more pulses of electrical stimulation each having a stimulation level equal to the initial stimulation level associated with stimulation event 1400. This stimulation level is represented in FIG. 14 by dashed line 1404. While three periods of electrical stimulation 1402 are shown to be included in stimulation event 1400, it will be recognized that any number of periods of electrical stimulation 1402 may be included in stimulation event 1400 as may serve a particular implementation.

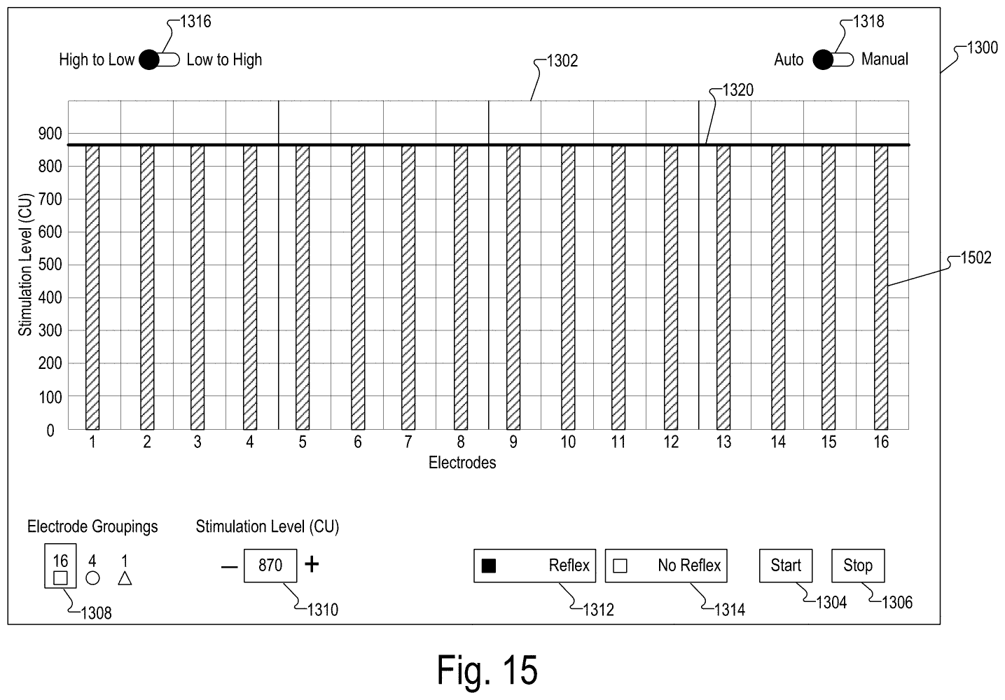

In some examples, diagnostic system 300 may graphically indicate, within one or more columns of graph 1302, an occurrence of each of the temporally spaced periods of electrical stimulation 1402. To illustrate, FIG. 15 shows vertical bars (e.g., vertical bar 1502) presented within each of the columns of graph 1302. Each of these vertical bars represents an occurrence of a period of electrical stimulation (e.g., one of periods of electrical stimulation 1402) included in a stimulation event (e.g., stimulation event 1400). Because a vertical bar is included in each of the columns of graph 1302, a user may quickly ascertain that the stimulation event is applied to each of the sixteen electrodes disposed on the electrode lead.

In some examples, the vertical bars may be displayed only during a period of electrical stimulation that is applied by the cochlear implant. Hence, if there are three periods of electrical stimulation during the stimulation event, the vertical bars may be displayed during three discreet and temporally separated time periods (e.g., in a flashing manner).

Diagnostic system 300 may be further configured to audibly indicate an occurrence of each of the periods of electrical stimulation 1402. For example, diagnostic system 300 may present sound (e.g., by way of one or more speakers that are included in or connected to diagnostic system 300) representative of the periods of electrical stimulation 1402. The sound may be presented in a synchronized manner with the presentation of the vertical bars representative of the periods of electrical stimulation 1402.

While the stimulation event having the initial stimulation level of 870 CU is applied by the cochlear implant to the electrode set, the user may monitor for an occurrence of a stapedial reflex. If the user does not provide, within a predetermined amount of time (e.g., a few seconds) subsequent to an occurrence of the stimulation event, user input indicating that no stapedial reflex occurred in response to the stimulation event, diagnostic system 300 may automatically determine that a stapedial reflex did occur in response to the stimulation event. Such user input may be provided by the user selecting option 1314 and/or in any other suitable manner.

The determination that a stapedial reflex occurred in response to the stimulation event is based on stimulation event sequence direction selector 1316 being set to high to low. Alternatively, as will be described below, if stimulation event sequence direction selector 1316 is set to low to high, diagnostic system 300 may automatically determine that that a stapedial reflex did not occur in response to the stimulation event unless user input to the contrary is received within the predetermined amount of time subsequent to the occurrence of the stimulation event.

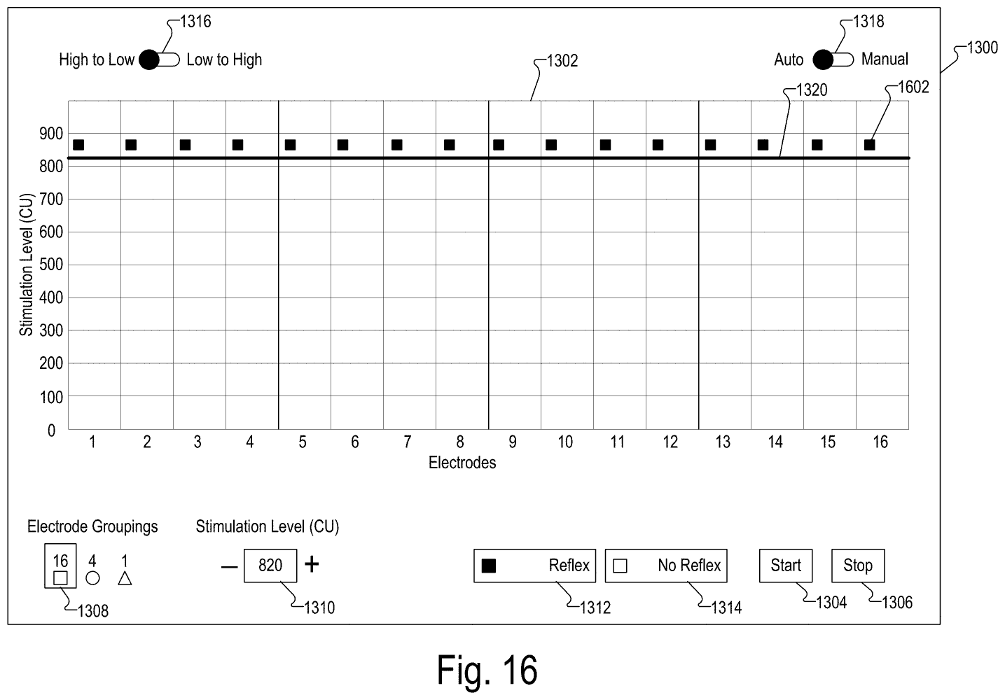

In response to the determination that the stapedial reflex occurred in response to the stimulation event, diagnostic system 300 may automatically present graphical markers within graph 1302 that indicate that the stapedial reflex occurred in response to the stimulation event. To illustrate, FIG. 16 shows a plurality of graphical markers (e.g., graphical marker 1602) presented within the columns of graph 1302. These graphical markers are positioned at the initial stimulation level (i.e., 870 CU) and include filled in squares to indicate that a stapedial reflex occurred in response to a stimulation event having the initial stimulation level.

Upon completion of the first stimulation event (i.e., the stimulation event that has the initial stimulation level), diagnostic system 300 may automatically direct the cochlear implant to apply a second stimulation event that has an incrementally decreased stimulation level compared to the initial stimulation level.

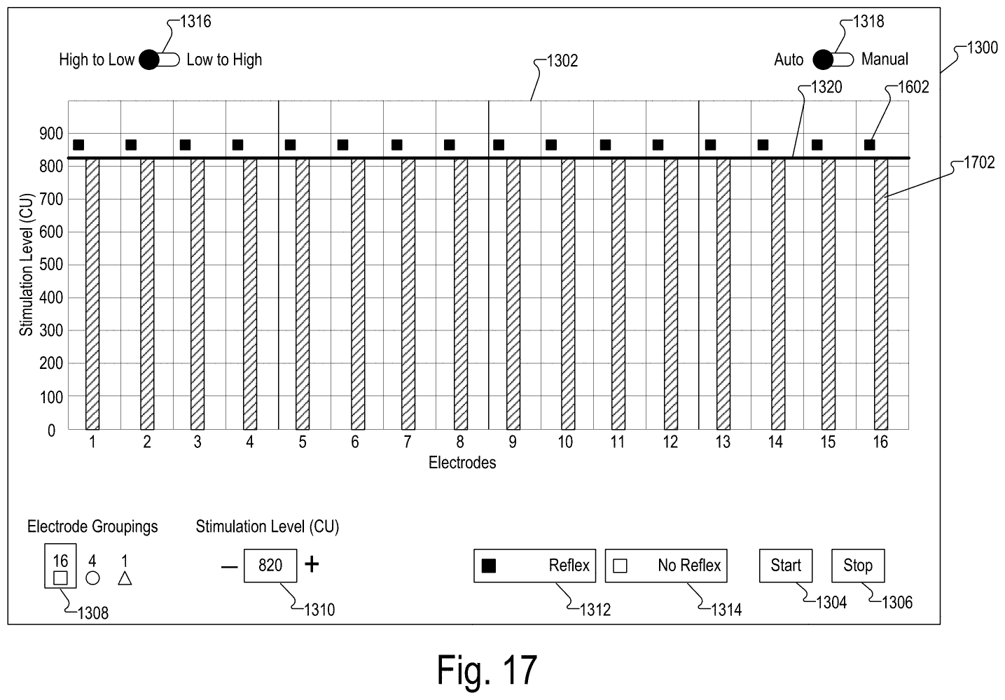

To illustrate, FIG. 16 shows that stimulation level indicator 1320 has been repositioned to be at 820 CU. The stimulation level step size between consecutive stimulation events may be set by the user, automatically by diagnostic system 300, and/or in any other suitable manner. Once the stimulation level for the second stimulation event has been set, diagnostic system 300 may direct the cochlear implant to apply the second stimulation event to the electrode set. A graphical representation of the second stimulation event may be displayed within the columns of graph 1302, as illustrated by the vertical bars (e.g., vertical bar 1702) shown in FIG. 17.

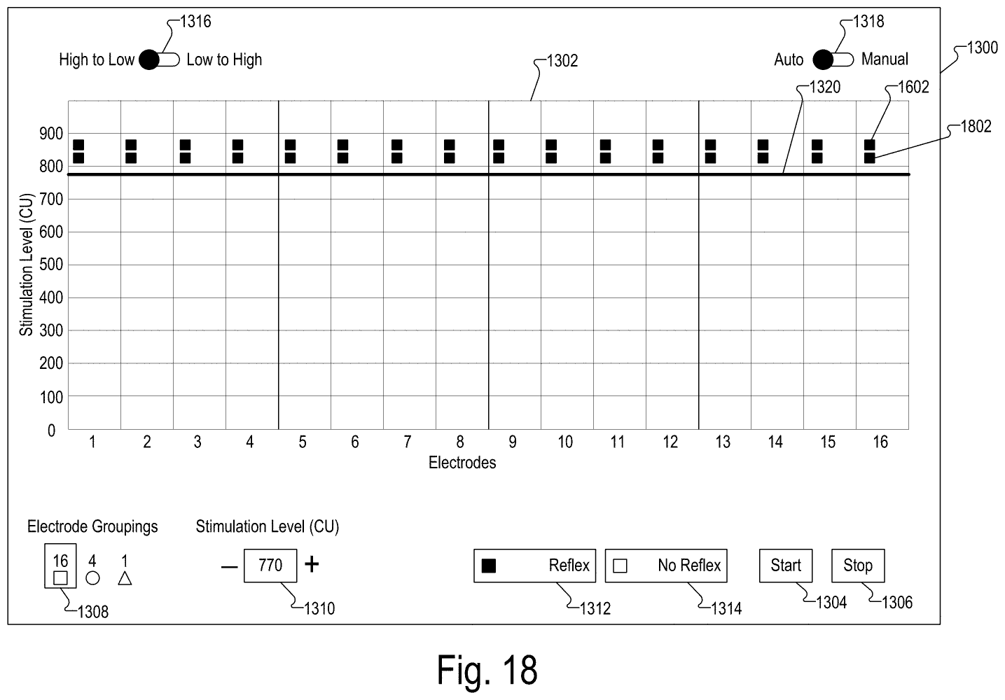

In this example, diagnostic system 300 again determines that a stapedial reflex occurred in response to the second stimulation event (e.g., by not receiving user input to the contrary). In response, as shown in FIG. 18, diagnostic system 300 automatically presents graphical markers (e.g., graphical marker 1802) within graph 1302 that indicate that the stapedial reflex occurred in response to the second stimulation event.

Upon completion of the second stimulation event, diagnostic system 300 may automatically direct the cochlear implant to apply a third stimulation event that has an incrementally decreased stimulation level compared to the stimulation level of the second stimulation event. To illustrate, FIG. 18 shows that stimulation level indicator 1320 has been repositioned to be at 770 CU.

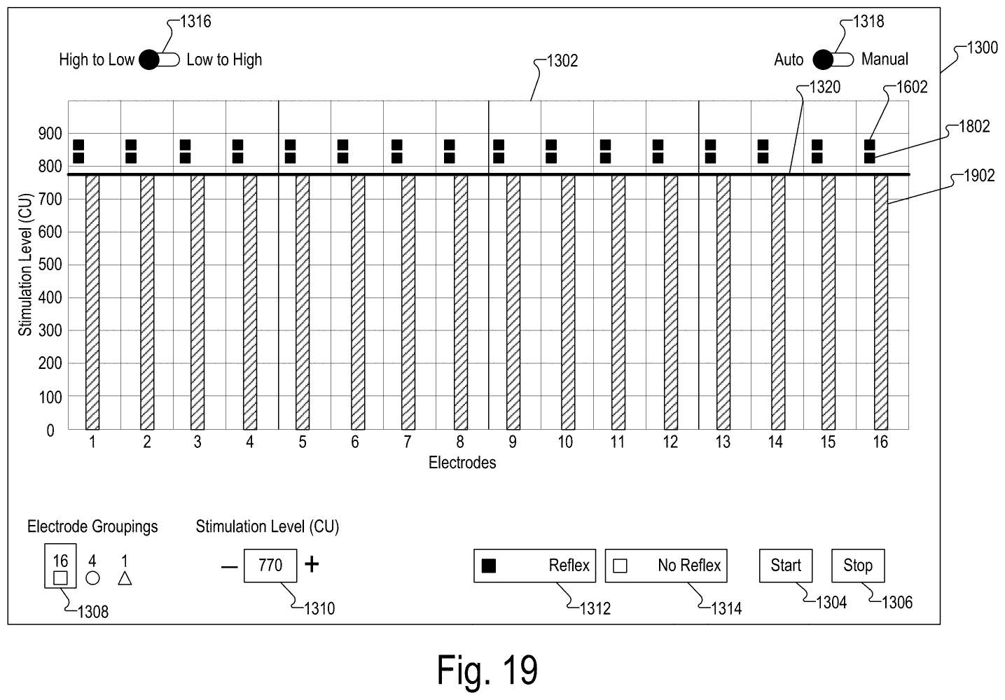

Once the stimulation level for the third stimulation event has been set, diagnostic system 300 may direct the cochlear implant to apply the third stimulation event to the electrode set. A graphical representation of the third stimulation event may be displayed within the columns of graph 1302, as illustrated by the vertical bars (e.g., vertical bar 1902) shown in FIG. 19.

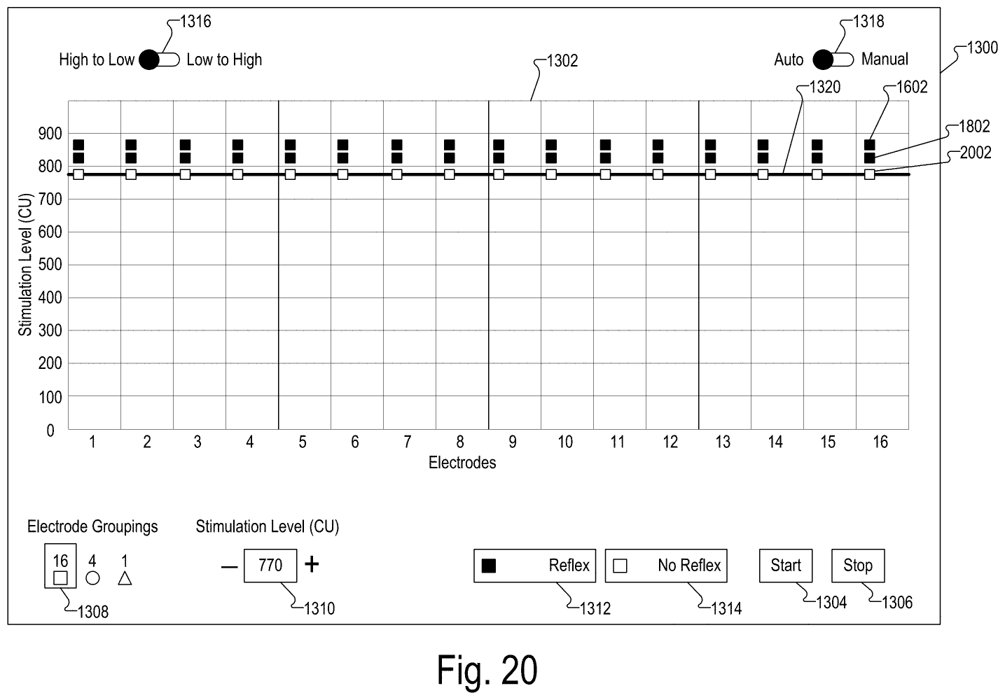

In this example, the user provides user input that a stapedial reflex did not occur in response to the third stimulation event. For example, the user may select option 1314. In response, diagnostic system 300 may direct the cochlear implant to stop applying the sequence of stimulation events. As shown in FIG. 20, diagnostic system 300 also automatically presents graphical markers (e.g., graphical marker 2002) within graph 1302 that indicate that a stapedial reflex did not occur in response to the third stimulation event. As shown, these graphical markers are visually distinct from the graphical markers that indicate that a stapedial reflex did occur in response to the first and second stimulation events.

In some examples, diagnostic system 300 may use the stimulation level of the stimulation event at which a change in the stapedial reflex state is detected to determine an ESRT for the recipient. For example, in the example just provided, diagnostic system 300 may designate a stimulation level of somewhere between 770 CU and 820 CU as the ESRT for the recipient. In some examples, diagnostic system 300 may store data representative of the ESRT and use this data to perform one or more fitting operations with respect to the recipient. For example, diagnostic system 300 may use the data representative of the ESRT to determine an M level for the recipient. In some examples, the stimulation level associated with the ESRT is designated as also being the M level for the recipient. Alternatively, the M level may be designated as being a certain amount above or below the stimulation level designated as being the ESRT. In some examples, diagnostic system 300 may program a sound processor associated with the cochlear implant to use the M level in a sound processing program that the sound processor uses to direct the cochlear implant to apply electrical stimulation representative of sounds presented to the recipient.

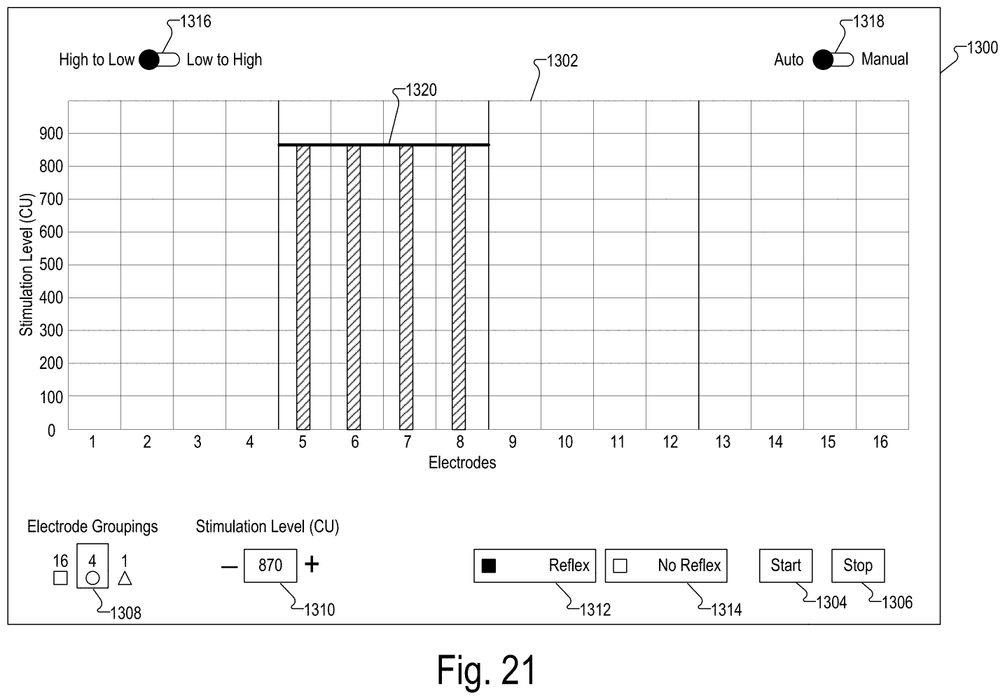

As mentioned, a user may select any number of electrodes to be included in the electrode set to which the stimulation events are applied. For example, FIG. 21 illustrates a configuration in which the user has selected an electrode grouping that includes only four electrodes for inclusion in the electrode set to which the stimulation events are applied. As shown in FIG. 21, electrode grouping selector 1308 is positioned over an electrode grouping number of four, thereby indicating that only four electrodes disposed on the electrode lead are to be included in the electrode set to which stimulation events are applied. The user may select which four electrodes are included in the electrode set in any suitable manner. For example, the user may select one or more columns included in graph 1302 to select four electrodes for inclusion in the electrode set. In the example of FIG. 21, electrodes 5 through 8 are included in the electrode set to which stimulation events are applied.

In some examples, diagnostic system 300 may automatically select the number of electrodes included in the electrode set to which the stimulation events are applied. For example, once a first sequence of stimulation events has been applied to an electrode set that includes all sixteen electrodes, diagnostic system 300 may automatically direct the cochlear implant to start applying a new sequence of stimulation events to an electrode set that includes only four electrodes. The new sequence of stimulation events may have an initial stimulation level equal to the stimulation level at which the first sequence of stimulation events stopped being applied. In this manner, a user may more easily determine a refined ESRT for progressively smaller sets of electrodes.

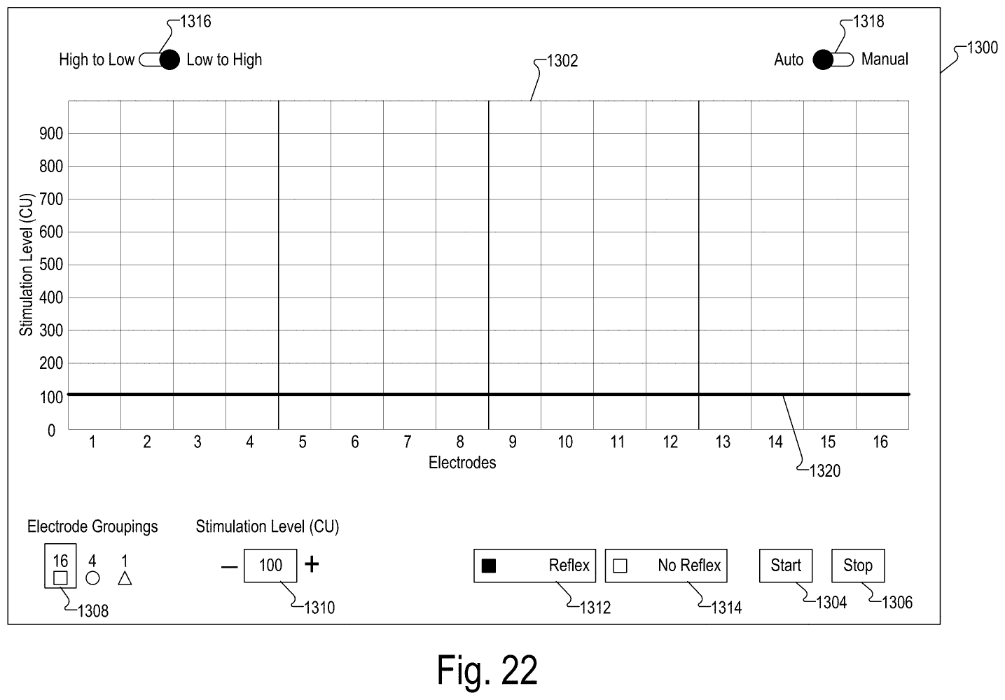

FIGS. 22-26 illustrate an exemplary ESRT measurement session that automatically and sequentially steps through a plurality of stimulation events having incrementally increasing stimulation levels. With reference to FIG. 22, a user may select start option 1304 to initiate the ESRT measurement session. In response to the user selection of start option 1304, diagnostic system 300 directs the cochlear implant to apply (e.g., concurrently) a first stimulation event to an electrode set that includes all sixteen electrodes disposed on an electrode lead that is implanted within a cochlea of a recipient. The first stimulation event has the initial stimulation level graphically indicated by stimulation level indicator 1320 and stimulation level adjustment field 1310. In the particular example of FIG. 22, this initial stimulation level is 100 CU.

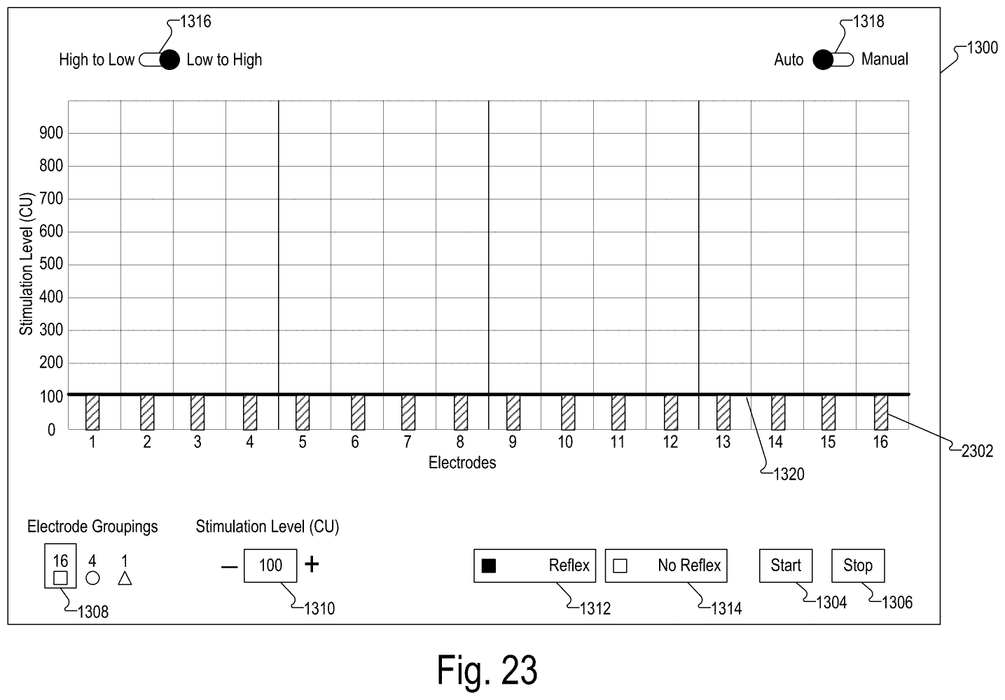

A graphical representation of the first stimulation event may be displayed within the columns of graph 1302, as illustrated by the vertical bars (e.g., vertical bar 2302) shown in FIG. 23. The graphical representation may be displayed in any suitable manner.

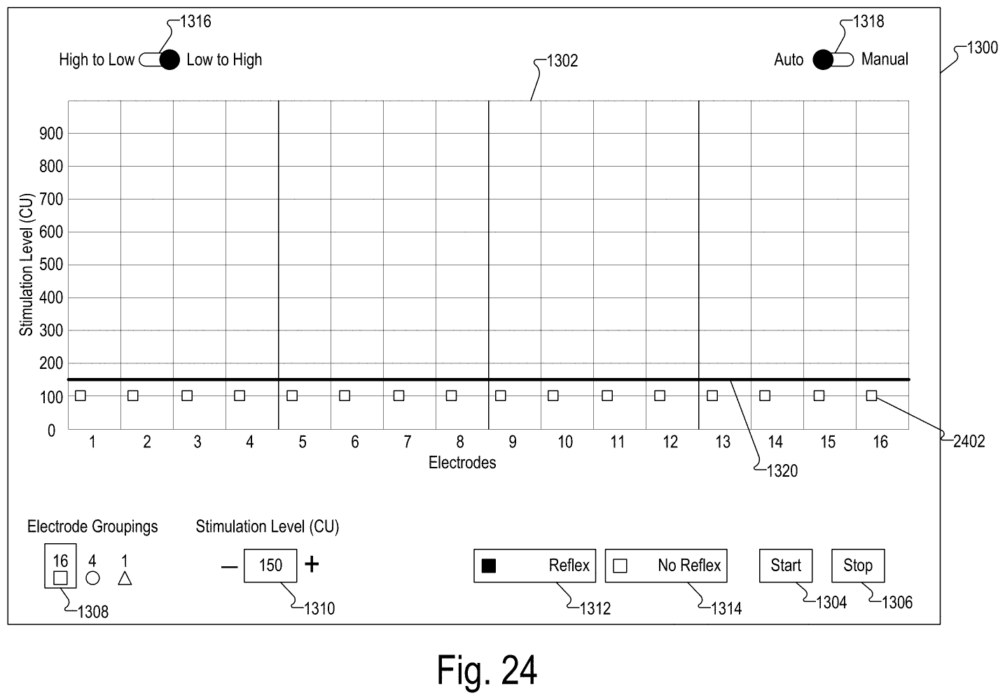

In this example, the user does not provide user input in response to the first stimulation event. Accordingly, diagnostic system 300 automatically determines that a stapedial reflex did not occur in response to the first stimulation event. In response, as shown in FIG. 24, diagnostic system 300 automatically presents graphical markers (e.g., graphical marker 2402) within graph 1302 that indicate that a stapedial reflex did not occur in response to the stimulation event.

Upon completion of the first stimulation event, diagnostic system 300 may automatically direct the cochlear implant to apply a second stimulation event that has an incrementally increased stimulation level compared to the second stimulation level. To illustrate, FIG. 24 shows that stimulation level indicator 1320 has been repositioned to be at 150 CU.

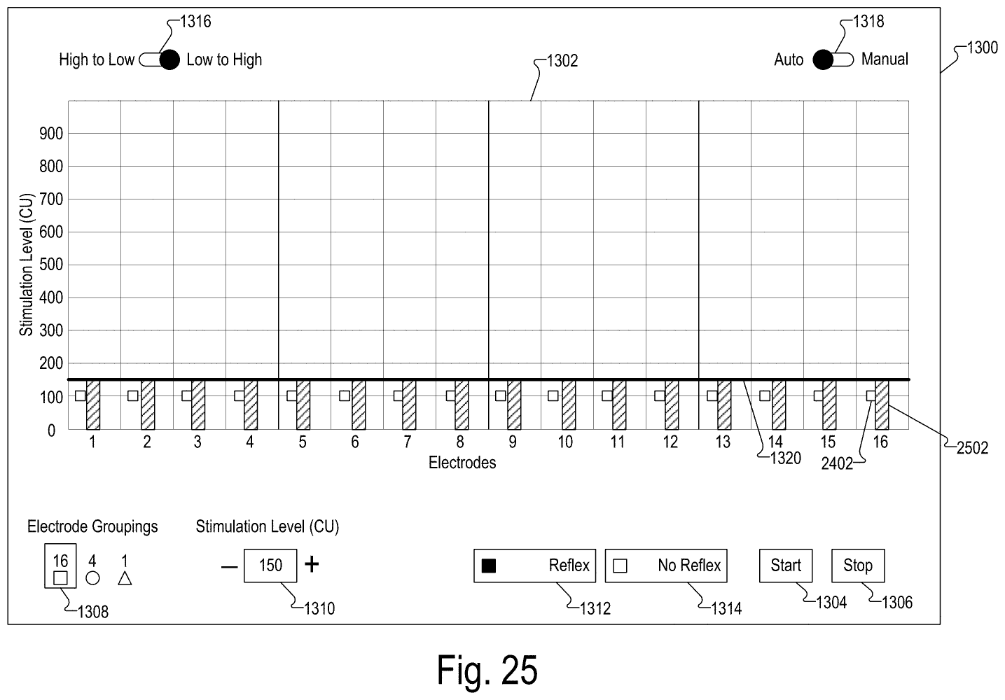

Once the stimulation level for the second stimulation event has been set, diagnostic system 300 may direct the cochlear implant to apply the second stimulation event to the electrode set. A graphical representation of the second stimulation event may be displayed within the columns of graph 1302, as illustrated by the vertical bars (e.g., vertical bar 2502) shown in FIG. 25.

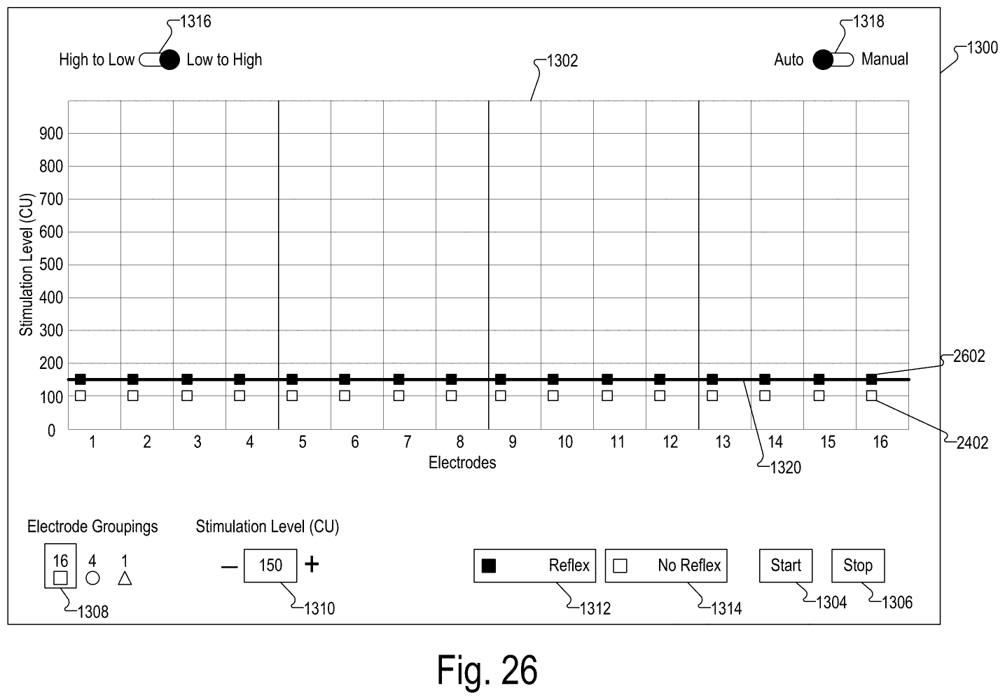

In this example, the user provides user input that a stapedial reflex did occur in response to the second stimulation event. For example, the user may select option 1312. In response, diagnostic system 300 may direct the cochlear implant to stop applying the sequence of stimulation events. As shown in FIG. 26, diagnostic system 300 also automatically presents graphical markers (e.g., graphical marker 2602) within graph 1302 that indicate that a stapedial reflex did occur in response to the second stimulation event. As shown, these graphical markers are visually distinct from the graphical markers that indicate that a stapedial reflex did not occur in response to the first stimulation event. Diagnostic system 300 may use the stimulation level associated with the second stimulation event to determine an ESRT and/or an M level for the recipient, as described above.

In some examples, diagnostic system 300 may perform an impedance test on the plurality of electrodes disposed on the electrode array prior to performing an ESRT measurement session. If diagnostic system 300 determines that a particular electrode included in the plurality of electrodes has an impedance that is not within a predetermined valid range, diagnostic system 300 may exclude the electrode from being included in the electrode set to which stimulation events are applied. For example, if an impedance of an electrode is too high or too low, it may mean that the electrode is either open or shorted. Hence, the electrode may be excluded from being included in the electrode set to which stimulation events are applied.

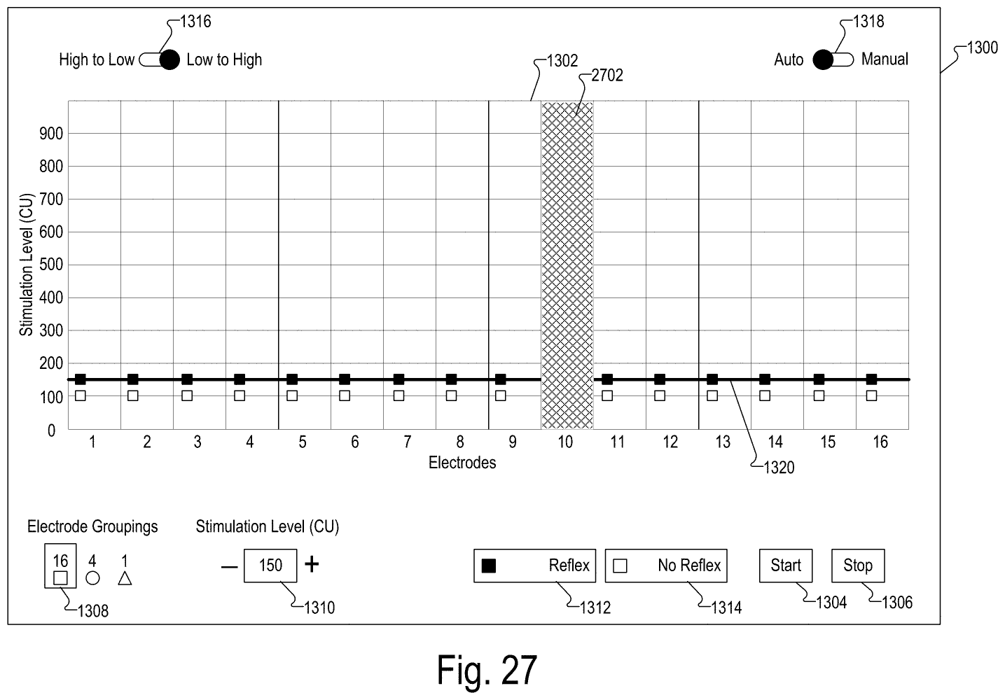

In some examples, an excluded electrode may be graphically identified within graphical user interface 1300. For example, FIG. 27 shows that the column for electrode 10 is filled in with a pattern 2702 that indicates that electrode 10 has an impedance that is out of valid range. In this manner, the user may readily ascertain that this electrode is not included in the electrode set to which stimulation events are applied.

Additional or alternative types of information may be presented within graphical user interface 1300 as may serve a particular implementation. For example, diagnostic system 300 may graphically indicate, within graphical user interface 1300, a compliance level associated with one or more electrodes. The compliance level may represent a maximum stimulation level that may be applied to the one or more electrodes.

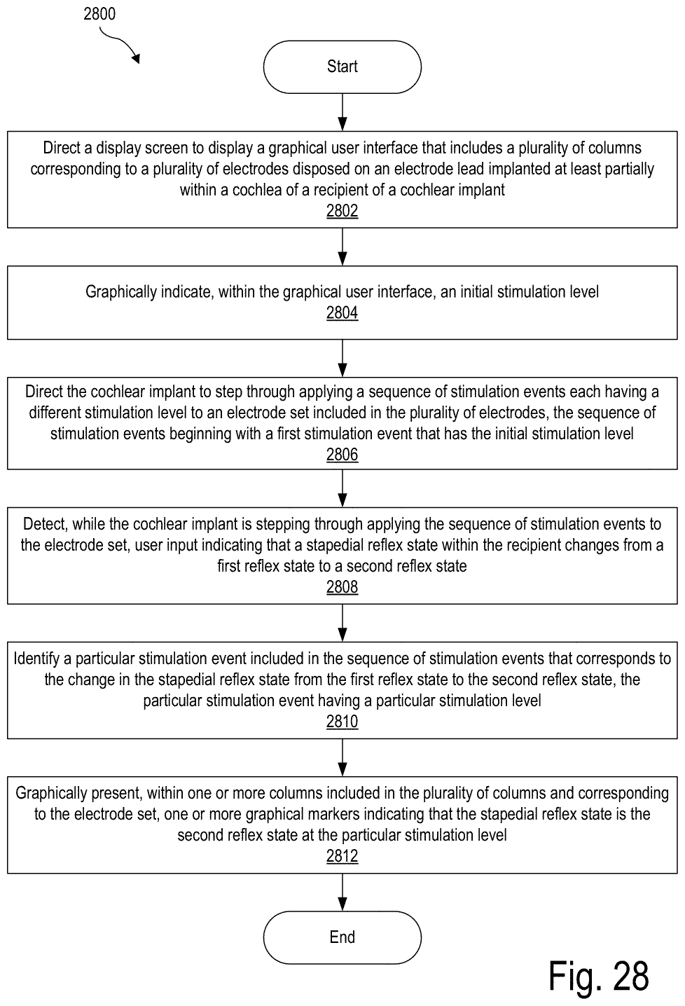

FIG. 28 illustrates an exemplary method 2800. The operations shown in FIG. 28 may be performed by diagnostic system 300 and/or any implementation thereof. While FIG. 28 illustrates exemplary operations according to one embodiment, other embodiments may omit, add to, reorder, and/or modify any of the operations shown in FIG. 28.

In operation 2802, a diagnostic system directs a display screen to display a graphical user interface that includes a plurality of columns corresponding to a plurality of electrodes disposed on an electrode lead implanted at least partially within a cochlea of a recipient of a cochlear implant. Operation 2802 may be performed in any of the ways described herein.

In operation 2804, the diagnostic system graphically indicates, within the graphical user interface, an initial stimulation level. Operation 2804 may be performed in any of the ways described herein.

In operation 2806, the diagnostic system directs the cochlear implant to step through applying a sequence of stimulation events each having a different stimulation level to an electrode set included in the plurality of electrodes, the sequence of stimulation events beginning with a first stimulation event that has the initial stimulation level. Operation 2806 may be performed in any of the ways described herein.

In operation 2808, the diagnostic system detects, while the cochlear implant is stepping through applying the sequence of stimulation events to the electrode set, user input indicating that a stapedial reflex state within the recipient changes from a first reflex state to a second reflex state. Operation 2808 may be performed in any of the ways described herein.

In operation 2810, the diagnostic system identifies a particular stimulation event included in the sequence of stimulation events that corresponds to the change in the stapedial reflex state from the first reflex state to the second reflex state, the particular stimulation event having a particular stimulation level. Operation 2810 may be performed in any of the ways described herein.

In operation 2812, the diagnostic system graphically presents, within one or more columns included in the plurality of columns and corresponding to the electrode set, one or more graphical markers indicating that the stapedial reflex state is the second reflex state at the particular stimulation level. Operation 2812 may be performed in any of the ways described herein.

In some examples, a non-transitory computer-readable medium storing computer-readable instructions may be provided in accordance with the principles described herein. The instructions, when executed by a processor of a computing device, may direct the processor and/or computing device to perform one or more operations, including one or more of the operations described herein. Such instructions may be stored and/or transmitted using any of a variety of known computer-readable media.

A non-transitory computer-readable medium as referred to herein may include any non-transitory storage medium that participates in providing data (e.g., instructions) that may be read and/or executed by a computing device (e.g., by a processor of a computing device). For example, a non-transitory computer-readable medium may include, but is not limited to, any combination of non-volatile storage media and/or volatile storage media. Exemplary non-volatile storage media include, but are not limited to, read-only memory, flash memory, a solid-state drive, a magnetic storage device (e.g. a hard disk, a floppy disk, magnetic tape, etc.), ferroelectric random-access memory (“RAM”), and an optical disc (e.g., a compact disc, a digital video disc, a Blu-ray disc, etc.). Exemplary volatile storage media include, but are not limited to, RAM (e.g., dynamic RAM).

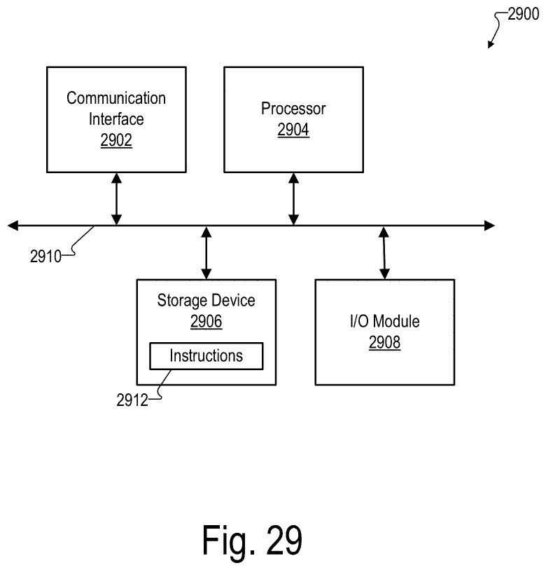

FIG. 29 illustrates an exemplary computing device 2900 that may be specifically configured to perform one or more of the processes described herein. As shown in FIG. 29, computing device 2900 may include a communication interface 2902, a processor 2904, a storage device 2906, and an input/output (“I/O”) module 2908 communicatively connected one to another via a communication infrastructure 2910. While an exemplary computing device 2900 is shown in FIG. 29, the components illustrated in FIG. 29 are not intended to be limiting. Additional or alternative components may be used in other embodiments. Components of computing device 2900 shown in FIG. 29 will now be described in additional detail.

Communication interface 2902 may be configured to communicate with one or more computing devices. Examples of communication interface 2902 include, without limitation, a wired network interface (such as a network interface card), a wireless network interface (such as a wireless network interface card), a modem, an audio/video connection, and any other suitable interface.

Processor 2904 generally represents any type or form of processing unit capable of processing data and/or interpreting, executing, and/or directing execution of one or more of the instructions, processes, and/or operations described herein. Processor 2904 may perform operations by executing computer-executable instructions 2912 (e.g., an application, software, code, and/or other executable data instance) stored in storage device 2906.

Storage device 2906 may include one or more data storage media, devices, or configurations and may employ any type, form, and combination of data storage media and/or device. For example, storage device 2906 may include, but is not limited to, any combination of the non-volatile media and/or volatile media described herein. Electronic data, including data described herein, may be temporarily and/or permanently stored in storage device 2906. For example, data representative of computer-executable instructions 2912 configured to direct processor 2904 to perform any of the operations described herein may be stored within storage device 2906. In some examples, data may be arranged in one or more databases residing within storage device 2906.

I/O module 2908 may include one or more I/O modules configured to receive user input and provide user output. I/O module 2908 may include any hardware, firmware, software, or combination thereof supportive of input and output capabilities. For example, I/O module 2908 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 2908 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 2908 is configured to provide graphical data to a display for presentation to a user. The graphical data may be representative of one or more graphical user interfaces and/or any other graphical content as may serve a particular implementation.

In some examples, any of the systems, computing devices, and/or other components described herein may be implemented by computing device 2900. For example, storage facility 302 may be implemented by storage device 2906, and processing facility 304 may be implemented by processor 2904.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. A system comprising:
a memory storing instructions;
a processor communicatively coupled to the memory and configured to execute the instructions to:
direct a display screen to display a graphical user interface that includes a plurality of columns corresponding to a plurality of electrodes disposed on an electrode lead implanted at least partially within a cochlea of a recipient of a cochlear implant;
graphically indicate, within the graphical user interface, an initial stimulation level;
direct the cochlear implant to step through applying a sequence of stimulation events each having a different stimulation level to an electrode set included in the plurality of electrodes, the sequence of stimulation events beginning with a first stimulation event that has the initial stimulation level;
detect, while the cochlear implant is stepping through applying the sequence of stimulation events to the electrode set, user input indicating that a stapedial reflex state within the recipient changes from a first reflex state to a second reflex state;
identify a particular stimulation event included in the sequence of stimulation events that corresponds to the change in the stapedial reflex state from the first reflex state to the second reflex state, the particular stimulation event having a particular stimulation level; and
graphically present, within one or more columns included in the plurality of columns and corresponding to the electrode set, one or more graphical markers indicating that the stapedial reflex state is the second reflex state at the particular stimulation level.

2. The system of claim 1, wherein:
the first reflex state represents a state in which a stapedial reflex occurs in response to one or more stimulation events; and
the second reflex state represents a state in which the stapedial reflex does not occur in response to one or more stimulation events.

3. The system of claim 2, wherein the directing of the cochlear implant to step through applying the sequence of stimulation events to the electrode set comprises directing the cochlear implant to:
apply, during a first time period, the first stimulation event that has the initial stimulation level; and
apply, during one or more time periods subsequent to the first time period, one or more additional stimulation events in order of incrementally decreasing stimulation levels.

4. The system of claim 3, wherein the processor is further configured to execute the instructions to graphically present, within the one or more columns corresponding to the electrode set, one or more graphical markers indicating that the stapedial reflex state is in the first reflex state at one or more stimulation levels associated with one or more stimulation events that are applied before the particular stimulation event having the particular stimulation level.

5. The system of claim 1, wherein:
the first reflex state represents a state in which a stapedial reflex does not occur in response to one or more stimulation events; and
the second reflex state represents a state in which the stapedial reflex occurs in response to one or more stimulation events.

6. The system of claim 5, wherein the directing of the cochlear implant to step through applying the sequence of stimulation events to the electrode set comprises directing the cochlear implant to:
apply, during a first time period, the first stimulation event that has the initial stimulation level; and
apply, during one or more time periods subsequent to the first time period, one or more additional stimulation events in order of incrementally increasing stimulation levels.

7. The system of claim 6, wherein the processor is further configured to execute the instructions to graphically present, within the one or more columns corresponding to the electrode set, one or more graphical markers indicating that the stapedial reflex state is in the first reflex state at one or more stimulation levels associated with one or more stimulation events that are applied before the particular stimulation event having the particular stimulation level.

8. The system of claim 1, wherein the graphically indicating of the initial stimulation level comprises presenting, within the graphical user interface, a graphical object representative of the initial stimulation level.

9. The system of claim 8, wherein the processor is further configured to execute the instructions to adjust a position of the graphical object as the cochlear implant steps through applying the sequence of stimulation events to the electrodes, wherein the position of the graphical object represents the stimulation levels of the stimulation events.

10. The system of claim 8, wherein the processor is further configured to execute the instructions to:
detect repositioning by a user of the graphical object; and
adjust the initial stimulation level based on the repositioning.

11. The system of claim 1, wherein the processor is further configured to execute the instructions to:
present a start option in the graphical user interface; and
perform the directing of the cochlear implant to step through applying the sequence of stimulation events in response to a selection by a user of the start option.

12. The system of claim 1, wherein the processor is further configured to execute the instructions to direct the cochlear implant to stop applying the sequence of stimulation events to the electrode set in response to the detecting of the user input indicating that the stapedial reflex state within the recipient changes from the first reflex state to the second reflex state.

13. The system of claim 1, wherein a stimulation event included in the sequence of stimulation events comprises a set number of temporally spaced periods of electrical stimulation applied at a stimulation level associated with the stimulation event.

14. The system of claim 13, wherein the processor is further configured to execute the instructions to graphically indicate, within the one or more columns, an occurrence of each of the temporally spaced periods of electrical stimulation.

15. The system of claim 13, wherein the processor is further configured to execute the instructions to audibly indicate an occurrence of each of the temporally spaced periods of electrical stimulation.

16. The system of claim 1, wherein the processor is further configured to execute the instructions to:
determine that an electrode included in the plurality of electrodes has an impedance that is not within a predetermined valid range; and
exclude the electrode from being included in the electrode set.

17. The system of claim 1, wherein the processor is further configured to execute the instructions to determine, based on the particular stimulation level, an electrical stapedial reflex threshold for the recipient.

18. The system of claim 1, wherein the processor is further configured to:
determine, based on the particular stimulation level, a most comfortable level for the recipient; and
program a sound processor communicatively coupled to the cochlear implant with the most comfortable level.

19. A diagnostic system comprising:
a computing module comprising:
a display screen, and
a processor configured to direct the display screen to display a graphical user interface that includes a plurality of columns corresponding to a plurality of electrodes disposed on an electrode lead implanted at least partially within a cochlea of a recipient of a cochlear implant; and
a base module configured to attach to the computing module and serve as a stand for the computing module, the base module housing an interface unit configured to be communicatively coupled to the processor and to the cochlear implant while the base module is attached to the computing module;
wherein the processor is further configured to:
graphically indicate, within the graphical user interface, an initial stimulation level;
direct the cochlear implant to step through applying a sequence of stimulation events each having a different stimulation level to an electrode set included in the plurality of electrodes, the sequence of stimulation events beginning with a first stimulation event that has the initial stimulation level;
detect, while the cochlear implant is stepping through applying the sequence of stimulation events to the electrode set, user input indicating that a stapedial reflex state within the recipient changes from a first reflex state to a second reflex state;
identify a particular stimulation event included in the sequence of stimulation events that corresponds to the change in the stapedial reflex state from the first reflex state to the second reflex state, the particular stimulation event having a particular stimulation level; and
graphically present, within one or more columns included in the plurality of columns and corresponding to the electrode set, one or more graphical markers indicating that the stapedial reflex state is the second reflex state at the particular stimulation level.

20. A method comprising:
directing, by a diagnostic system, a display screen to display a graphical user interface that includes a plurality of columns corresponding to a plurality of electrodes disposed on an electrode lead implanted at least partially within a cochlea of a recipient of a cochlear implant;
graphically indicating, by the diagnostic system within the graphical user interface, an initial stimulation level;
directing, by the diagnostic system, the cochlear implant to step through applying a sequence of stimulation events each having a different stimulation level to an electrode set included in the plurality of electrodes, the sequence of stimulation events beginning with a first stimulation event that has the initial stimulation level;
detecting, by the diagnostic system while the cochlear implant is stepping through applying the sequence of stimulation events to the electrode set, user input indicating that a stapedial reflex state within the recipient changes from a first reflex state to a second reflex state;
identifying, by the diagnostic system, a particular stimulation event included in the sequence of stimulation events that corresponds to the change in the stapedial reflex state from the first reflex state to the second reflex state, the particular stimulation event having a particular stimulation level; and
graphically presenting, by the diagnostic system within one or more columns included in the plurality of columns and corresponding to the electrode set, one or more graphical markers indicating that the stapedial reflex state is the second reflex state at the particular stimulation level.
